SEQUENCE LISTING (58 sequences)

SEQ ID NO: 1 - protein, 20 aa, synthetic construct
  REGION 1..20: note = Leader peptide
MSRSVALAVLALLSLSGLEA

SEQ ID NO: 2 - protein, 10 aa, synthetic construct
  REGION 1..10: note = Peptide antigen
ALWGPDPAAA

SEQ ID NO: 3 - protein, 15 aa, synthetic construct
  REGION 1..15: note = First Linker
GGGGSGGGGSGGGGS

SEQ ID NO: 4 - protein, 15 aa, synthetic construct
  REGION 1..15: note = First Linker
GCGASGGGGSGGGGS

SEQ ID NO: 5 - protein, 99 aa, synthetic construct [sequence omitted]

SEQ ID NO: 6 - protein, 20 aa, synthetic construct
  REGION 1..20: note = Second Linker
GGGGSGGGGSGGGGSGGGGS

SEQ ID NO: 7 - protein, 182 aa, synthetic construct
  REGION 1..182: note = [Alpha] 1 & 2 domain derived from human HLA-G
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRA

SEQ ID NO: 8 - protein, 182 aa, synthetic construct
  REGION 1..182: note = Human HLA-A2 [alpha]1 & 2 domain
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRT

SEQ ID NO: 9 - protein, 113 aa, synthetic construct
  REGION 1..113: note = HLA-G [alpha]3
DPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 10 - protein, 11 aa, synthetic construct
  REGION 1..11: note = Factor Xa restriction site
IEGRTGTKLGP

SEQ ID NO: 11 - protein, 13 aa, synthetic construct
  REGION 1..13: note = SpotTag
PDRVRAVSHWSSC

SEQ ID NO: 12 - protein, 10 aa, synthetic construct
  REGION 1..10: note = Myc tag
EQKLISEEDL

SEQ ID NO: 13 - protein, 6 aa, synthetic construct
  REGION 1..6: note = His tag
HHHHHH

SEQ ID NO: 14 - protein, 5 aa, synthetic construct
  REGION 1..5: note = Spacer sequence
NSAVD

SEQ ID NO: 15 - protein, 473 aa, synthetic construct
  REGION 1..473: note = Recombinant polypeptide
MSRSVALAVLALLSLSGLEAFLIEEIERLGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 16 - protein, 444 aa, synthetic construct
  REGION 1..444: note = Recombinant polypeptide
GCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 17 - protein, 650 aa, synthetic construct
  REGION 1..650: note = Receptor ILT2 (also known as LILRB1)
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 18 - protein, 598 aa, synthetic construct
  REGION 1..598: note = Receptor ILT4
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 19 - protein, 110 aa, synthetic construct
  REGION 1..110: note = Human insulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 20 - protein, 21 aa, synthetic construct
  REGION 1..21: note = Human HLA-G [alpha]3 C-terminal amino acid sequence from intron 4
SKEGDGGIMSVRESRSLSEDL

SEQ ID NO: 21 - protein, 92 aa, synthetic construct
  REGION 1..92: note = Human HLA-G [alpha]3 without C-terminal amino acid sequence fromintron 4
DPPKTHVTHHPVFDYEATLRCWALGFYPAEIILTWQRDGEDQTQDVELVETRPAGDGTFQKWAAVVVPSGEEQRYTCHVQHEGLPEPLMLRW

SEQ ID NO: 22 - protein, 9 aa, synthetic construct
  REGION 1..9: note = Peptide antigen
ILKDFSILL

SEQ ID NO: 23 - protein, 9 aa, synthetic construct
  REGION 1..9: note = Peptide antigen
EWESNGQPE

SEQ ID NO: 24 - protein, 9 aa, synthetic construct
  REGION 1..9: note = Peptide antigen
KIADPICTF

SEQ ID NO: 25 - protein, 10 aa, synthetic construct
  REGION 1..10: note = Peptide antigen
LALWGPDPAA

SEQ ID NO: 26 - protein, 10 aa, synthetic construct
  REGION 1..10: note = Peptide antigen
WYIPPSLRTL

SEQ ID NO: 27 - protein, 9 aa, synthetic construct
  REGION 1..9: note = Peptide antigen
RMMEYGTTM

SEQ ID NO: 28 - protein, 86 aa, synthetic construct
  REGION 1..86: note = Proinsulin
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 29 - protein, 30 aa, synthetic construct
  REGION 1..30: note = Mature insulin (B chain)
FVNQHLCGSHLVEALYLVCGERGFFYTPKT

SEQ ID NO: 30 - protein, 25 aa, synthetic construct
  REGION 1..25: note = Mature insulin (A chain)
LQKRGIVEQCCTSICSLYQLENYCN

SEQ ID NO: 31 - protein, 585 aa, synthetic construct
  REGION 1..585: note = Human Glutamate decarboxylase 65 (GAD65)
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 32 - protein, 89 aa, synthetic construct
  REGION 1..89: note = Human Islet amyloid polypeptide
MGILKLQVFLIVLSVALNHLKATPIESHQVEKRKCNTATCATQRLANFLVHSSNNFGAILSSTNVGSNTYGKRNAVEVLKREPLNYLPL

SEQ ID NO: 33 - protein, 369 aa, synthetic construct
  REGION 1..369: note = Human Zinc transporter 8
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 34 - protein, 452 aa, synthetic construct
  REGION 1..452: note = Recombinant polypeptide
SIINFEKLGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 35 - protein, 452 aa, synthetic construct
  REGION 1..452: note = Recombinant polypeptide
AVYNFATMGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 36 - protein, 455 aa, synthetic construct
  REGION 1..455: note = Recombinant polypeptide
FSRVVHLYRNGGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSMRYFETAVSRPGLEEPRYISVGYVDNKEFVRFDSDAENPRYEPRAPWMEQEGPEYWERETQKAKGQEQWFRVSLRNLLGCYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 37 - protein, 454 aa, synthetic construct
  REGION 1..454: note = Recombinant polypeptide
VGWYRSPFSRGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSMRYFETAVSRPGLEEPRYISVGYVDNKEFVRFDSDAENPRYEPRAPWMEQEGPEYWERETQKAKGQEQWFRVSLRNLLGCYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 38 - protein, 9 aa, synthetic construct
  REGION 1..9: note = Peptide antigen
KVDVNYAFL

SEQ ID NO: 39 - protein, 9 aa, synthetic construct
  REGION 1..9: note = Peptide antigen
KQKGFVPFL

SEQ ID NO: 40 - protein, 9 aa, synthetic construct
  REGION 1..9: note = Peptide antigen
SYDTGDKAL

SEQ ID NO: 41 - protein, 9 aa, synthetic construct
  REGION 1..9: note = Peptide antigen
GILKLQVFL

SEQ ID NO: 42 - protein, 9 aa, synthetic construct
  REGION 1..9: note = Peptide antigen
FLIVLSVAL

SEQ ID NO: 43 - protein, 9 aa, synthetic construct
  REGION 1..9: note = Peptide antigen
VLSVALNHL

SEQ ID NO: 44 - protein, 9 aa, synthetic construct
  REGION 1..9: note = Peptide antigen
REPLNYLPL

SEQ ID NO: 45 - protein, 9 aa, synthetic construct
  REGION 1..9: note = Peptide antigen
ALWMRLLPL

SEQ ID NO: 46 - protein, 9 aa, synthetic construct
  REGION 1..9: note = Peptide antigen
RLLPLLALL

SEQ ID NO: 47 - protein, 10 aa, synthetic construct
  REGION 1..10: note = Peptide antigen
SHLVEALYLV

SEQ ID NO: 48 - protein, 9 aa, synthetic construct
  REGION 1..9: note = Peptide antigen
HLVEALYLV

SEQ ID NO: 49 - protein, 9 aa, synthetic construct
  REGION 1..9: note = Peptide antigen
SLQPLALEG

SEQ ID NO: 50 - protein, 10 aa, synthetic construct
  REGION 1..10: note = Peptide antigen
SLQKRGIVEQ

SEQ ID NO: 51 - protein, 10 aa, synthetic construct
  REGION 1..10: note = Peptide antigen
GIVEQCCTSI

SEQ ID NO: 52 - protein, 9 aa, synthetic construct
  REGION 1..9: note = Peptide antigen
SLYQLENYC

SEQ ID NO: 53 - protein, 9 aa, synthetic construct
  REGION 1..9: note = Peptide antigen
LLIDLTSFL

SEQ ID NO: 54 - protein, 9 aa, synthetic construct
  REGION 1..9: note = Peptide antigen
KPPSKRLTF

SEQ ID NO: 55 - protein, 10 aa, synthetic construct
  REGION 1..10: note = Peptide antigen
AVAANIVLTV

SEQ ID NO: 56 - protein, 9 aa, synthetic construct
  REGION 1..9: note = Peptide antigen
VLASTITIL

SEQ ID NO: 57 - protein, 10 aa, synthetic construct
  REGION 1..10: note = Peptide antigen
ILAVDGVLSV

SEQ ID NO: 58 - protein, 8 aa, synthetic construct
  REGION 1..8: note = Peptide antigen
AVYNFATM

(19) United States
(12) Patent Application Publication
(10) Pub. No.: US 2025/0205323 A1
(43) Pub. Date: Jun. 26, 2025

(54) MHC IB-MEDIATED ISLET-ANTIGEN-SPECIFIC IMMUNOSUPPRESSION AS A NOVEL TREATMENT FOR TYPE 1 DIABETES

(71) Applicant: Julius-Maximilians-Universität Würzburg, Würzburg (DE)

(72) Inventors: Valentin BRUTTEL, Würzburg (DE); Jörg WISCHHUSEN, Würzburg (DE); Daniela BRÜNNERT, Würzburg (DE); Fadhil AHSAN, Berlin (DE)

(73) Assignee: Julius-Maximilians-Universität Würzburg, Würzburg (DE)

(21) Appl. No.: 18/849,139

(22) PCT Filed: Mar. 24, 2023
(86) PCT No.: PCT/EP2023/057681

§ 371 Date: Sep. 20, 2024

(30) Foreign Application Priority Data
Mar. 24, 2022 (EP) 22164163.2

(51) Int. Cl. A61K 39/00; A61K 38/00; A61K 39/385; A61P 3/10; A61P 37/04; C07K 14/47; C07K 14/62; C07K 14/74; C12N 9/88

(52) U.S. Cl. CPC A61K 39/0008; A61K 39/385; A61P 3/10; A61P 37/04; C07K 14/4711; C07K 14/62; C07K 14/70539; C12N 9/88; A61K 38/00; A61K 2039/605; C07K 2319/00; C12Y 401/01015

(57) ABSTRACT

The present invention relates to therapeutical uses of non-classical human major histocompatibility complex (MHC) molecules (also named MHC class Ib molecules) in combination with peptide antigens for the treatment of type 1 diabetes (T1D). The invention more specifically relates to recombinant polypeptides comprising peptide antigens and one or more domains of a non-classical MHC class Ib molecule. The invention also relates to methods of producing such recombinant polypeptides, pharmaceutical compositions comprising the same, as well as their uses for treating type 1 diabetes (T1D).

### FIELD OF THE INVENTION

The present invention relates to therapeutical uses of non-classical human major histocompatibility complex (MHC) molecules (also named MHC class Ib molecules) in combination with peptide antigens for the treatment of type 1 diabetes (T1D). The invention more specifically relates to recombinant polypeptides comprising peptide antigens and one or more domains of a non-classical MHC class Ib molecule. The invention also relates to methods of producing such recombinant polypeptides, pharmaceutical compositions comprising the same, as well as their uses for treating type 1 diabetes (T1D).

### BACKGROUND

As in all autoimmune diseases, an excessive immune reaction against the body's own tissue, which is mistakenly recognized as foreign and attacked, leads to type 1 diabetes (T1D). T cells, which can recognize individual target structures (antigens) very selectively due to their receptors, play a decisive role here. Currently, such diseases are mainly treated with anti-inflammatory drugs or antibodies, which systemically inhibit immune responses and thus dampen symptoms or slow down the progression of diseases. At the same time, however, functioning T cells in particular are also essential for the survival of patients with autoimmune diseases, as they are able to recognize and combat dangerous viruses, bacteria, parasites and mutated cells. Systemic immunosuppression can therefore only be used in a narrow therapeutic window. Thus, in the case of T1D, one attempt is to partially compensate for defects that have developed, by administering insulin in type 1 diabetes. However, blood glucose levels monitoring and accurate dosing of insulin is very difficult. Thus, the unmet medical need is very high, T1D patients have a life expectancy reduced by 11-13 years due to numerous sequelae (Livingstone et al, JAMA. 2015 Jan. 6; 313(1):37-44).

CD8 T cells that attack islet cells play a crucial role in T1D (Tsai S, Shameli A, Santamaria P. CD8+ T cells in type 1 diabetes. Adv Immunol. 2008; 100:79-124.)

However, one of the key diagnostic tools for T1D is testing serum for autoantibodies such as Islet cell cytoplasmic autoantibodies (ICA), Glutamic acid decarboxylase autoantibodies (GADA), Insulinoma-associated-2 autoantibodies (IA-2A) or Insulin autoantibodies (IAA). This way, at risk patients can be identified before a significant destruction of islet cells occurs. Furthermore, inhibition of both CD8 T cell responses and autoantibody formation could significantly improve disease outcome.

WO 2018/215340 relates to combinations of MHC class Ib molecules and peptides for targeted therapeutic immunomodulation.

Taken together, there remains a need for improved drugs for the treatment of type 1 diabetes (T1D).

### DESCRIPTION OF THE INVENTION

The inventors have found that human MHC class Ib molecules such as HLA-G possess the ability to induce antigen-specific tolerance towards presented peptide antigens. Thus, albeit being of similar structure and sequence as classical human MHC class Ia molecules which induce antigen peptide-specific immune responses, MHC class Ib molecules can advantageously be used according to the invention to suppress immune responses in an antigen-specific manner. Additionally, the inventors have found that for the suppression of immune responses according to the invention, molecules other than naturally occurring MHC class Ib molecules, and in particular polypeptides which only comprise at least one domain of an MHC class Ib molecule, preferably at least an [alpha]3 domain of an MHC class Ib molecule, can be used: The [alpha]1 and [alpha]2 domains of variable class I a molecules can be combined with the [alpha]3 domain of a human MHC class Ib molecule in order to suppress immune responses towards peptides presented by these antigens. The inventors further found that the antigen which is accommodated in the peptide-binding cleft of HLA-G induces selective tolerance in cognate T cells. The inventors observed, inter alia, two mechanisms: induction of apoptosis in highly activated cytotoxic CD8*T cells, and induction of regulatory T cells in cognate naïve T cells. Accordingly, the invention allows to induce selective tolerance induction towards specific antigens without compromising protective immune responses against pathogens.

Antigen-loaded HLA-G molecules can be unstable. Thus, the inventors designed soluble recombinant polypeptides comprising a peptide antigen, an MHC class Ib molecule such as HLA-G and 32-microglobulin (b2m), and connected these three components covalently (e.g., via covalent linkers). Alternatively, the antigen-binding a1 and a2 domains of an MHC class Ib molecule such as HLA-G were exchanged by the respective domains of other MHC molecules to enhance the flexibility and versatility of these recombinant polypeptides (see, for instance, FIG. 2). These alternative recombinant polypeptides can be designed with antigen-binding domains of other human HLA molecules. It was previously found that constructs comprising the a1 and a2 domains of murine H2-K^{b} can present the ovalbumin-derived peptide SIINFEKL to OT-1 T cells. (OT-1 T cells express a transgenic T cell receptor that specifically recognizes this antigen) (WO 2018/215340).

Surprisingly, the inventors have found that by using the recombinant polypeptides of the invention, immune responses against human human proinsulin/human insulin (INS), human Glutamate decarboxylase 65 (GAD65), human islet amyloid polypeptide (IAPP) and human Zinc transporter 8 (ZNT8) can be suppressed. Thus, according to the invention, type 1 diabetes (T1D) can be treated by the recombinant polypeptides of the invention.

Moreover, according to the invention, the recombinant polypeptides of the invention do not only modulate T-cell responses but also prevent the formation of autoantibodies to human proinsulin and human insulin (INS), human Glutamate decarboxylase 65 (GAD65), human islet amyloid polypeptide (IAPP) and human Zinc transporter 8 (ZNT8). It is expected that this advantage will contribute to a clinical improvement in human patients having type 1 diabetes (T1D), because such autoantibodies are, besides CD8+ T cells, involved in the pathology of type 1 diabetes (T1D).

Accordingly, the invention relates to the following preferred embodiments:
1. A recombinant polypeptide capable of presenting a peptide antigen, the recombinant polypeptide comprising, in an N- to C-terminal order,
   i) a peptide antigen presented by said recombinant polypeptide, wherein the peptide antigen is a peptide of human proinsulin or human insulin, human Glutamate decarboxylase 65, human islet amyloid polypeptide or human Zinc transporter 8;
   ii) optionally a linker sequence;
   iii) optionally a sequence of a human polypeptide domain comprising a sequence of a human 132 microglobulin, or an amino acid sequence at least 90% identical to the amino acid sequence of human 132 microglobulin represented by SEQ ID NO: 5;
   iv) optionally a linker sequence;
   v) optionally an [alpha]1 domain of an MHC molecule;
   vi) optionally an [alpha]2 domain of an MHC molecule;
   vii) an [alpha]3 domain of an MHC class Ib molecule or a derivative of an [alpha]3 domain of an MHC class Ib molecule, said derivative being capable of binding to ILT2 or ILT4;
   viii) optionally a protease cleavage site;
   ix) optionally a spacer sequence; and
   x) optionally an affinity tag.
2. The recombinant polypeptide according to item 1, wherein said peptide antigen according to i) is 7 to 11 amino acids in length, preferably 8-10 amino acids in length.
3. The recombinant polypeptide according to item 1 or 2, wherein said peptide antigen according to
   i) consists of an amino acid sequence selected from the group consisting of the amino acid sequences of SEQ ID NO: 2, SEQ ID NO: 22, SEQ ID NO: 23, SEQ ID NO: 24, SEQ ID NO: 25, SEQ ID NO: 26 and SEQ ID NO: 27.
4. The recombinant polypeptide according to any one of the preceding items, wherein said peptide antigen consists of an amino acid sequence selected from the group consisting of the amino acid sequences of SEQ ID NO: 2, SEQ ID NO: 22, and SEQ ID NO: 23.
5. The recombinant polypeptide according to any one of items 1-3, wherein said peptide antigen is a peptide antigen of human proinsulin or human insulin and preferably consists of an amino acid sequence selected from the group consisting of SEQ ID NO: 2 and SEQ ID NO: 25.
6. The recombinant polypeptide according to any one of items 1-3, wherein said peptide antigen is a peptide antigen of human Glutamate decarboxylase 65 and preferably consists of an amino acid sequence selected from the group consisting of SEQ ID NO: 23, SEQ ID NO: 26 and SEQ ID NO: 27.
7. The recombinant polypeptide according to any one of items 1-3, wherein said peptide antigen is a peptide antigen of human Glutamate decarboxylase 65 and consists of the amino acid sequence of SEQ ID NO: 26.
8. The recombinant polypeptide according to any one of items 1-3, wherein said peptide antigen is a peptide antigen of human Zinc transporter 8 and preferably consists of an amino acid sequence selected from the group consisting of SEQ ID NO: 22 and SEQ ID NO: 24.
9. The recombinant polypeptide according to any one of items 1-3, wherein said peptide antigen is a peptide antigen of human islet amyloid polypeptide.
10. The recombinant polypeptide according to any one of the preceding items, wherein said [alpha]1 domain according to (v) and said [alpha]2 domain according to (vi) are from a human MHC class Ia molecule or from a human MHC class Ib molecule.
11. The recombinant polypeptide according to item 10, wherein said [alpha]1 domain according to (v) and said [alpha]2 domain according to (vi) are from a human MHC class Ia molecule.
12. The recombinant polypeptide according to item 11, wherein said [alpha]1 domain according to (v) and said [alpha]2 domain according to (vi) are from a human HLA-A2 molecule.
13. The recombinant polypeptide according to item 10, wherein said [alpha]1 domain according to (v) and said [alpha]2 domain according to (vi) are from a human MHC class Ib molecule.
14. The recombinant polypeptide according to item 13, wherein said [alpha]1 domain according to (v) and said [alpha]2 domain according to (vi) are from a human HLA-G molecule.
15. The recombinant polypeptide according to any one of the preceding items, wherein the [alpha]3 domain of the MHC class Ib molecule according to (vii) is an [alpha]3 domain of human HLA-E, human HLA-F or human HLA-G.
16. The recombinant polypeptide according to any one of the preceding items, wherein the [alpha]3 domain of the MHC class Ib molecule according to (vii) is an [alpha]3 domain of human HLA-G.
17. The recombinant polypeptide according to any one of the preceding items, wherein the [alpha]3 domain or derivative according to (vii) is identical to or has at least 80% amino acid sequence identity, preferably at least 90% amino acid sequence identity, with the [alpha]3 domain having the amino acid sequence of SEQ ID NO: 9 or SEQ ID NO: 21.
18. The recombinant polypeptide according to item 17, wherein the [alpha]3 domain or derivative according to (vii) is identical to or has at least 92% amino acid sequence identity with the [alpha]3 domain having the amino acid sequence of SEQ ID NO: 9 or SEQ ID NO: 21.
19. The recombinant polypeptide according to item 17, wherein the [alpha]3 domain or derivative according to (vii) is identical to or has at least 94% amino acid sequence identity with the [alpha]3 domain having the amino acid sequence of SEQ ID NO: 9 or SEQ ID NO: 21.
20. The recombinant polypeptide according to item 17, wherein the [alpha]3 domain or derivative according to (vii) is identical to or has at least 96% amino acid sequence identity with the [alpha]3 domain having the amino acid sequence of SEQ ID NO: 9 or SEQ ID NO: 21.
21. The recombinant polypeptide according to item 17, wherein the [alpha]3 domain or derivative according to (vii) is identical to or has at least 98% amino acid sequence identity with the [alpha]3 domain having the amino acid sequence of SEQ ID NO: 9 or SEQ ID NO: 21.
22. The recombinant polypeptide according to item 17, wherein the [alpha]3 domain or derivative according to (vii) is identical to or has at least 99% amino acid sequence identity with the [alpha]3 domain having the amino acid sequence of SEQ ID NO: 9 or SEQ ID NO: 21.
23. The recombinant polypeptide according to item 17, wherein the [alpha]3 domain according to (vii) is identical to the [alpha]3 domain having the amino acid sequence of SEQ ID NO: 9 or SEQ ID NO: 21.
24. The recombinant polypeptide according to any one of the preceding items, wherein the linker sequence according to (ii) and/or the linker sequence according to (iv) comprises the amino acid sequence (GGGGS)n, wherein n is an integer equal to or higher than 1.
25. The recombinant polypeptide according to item 24, wherein the linker sequence according to (ii) comprises the amino acid sequence (GGGGS)n, and wherein n is an integer selected from the group consisting of 1, 2, 3, 4, 5, 6, 7, 8, 9 and 10 and is preferably selected from the group consisting of 2, 3, 4 and 5.
26. The recombinant polypeptide according to item 24 or 25, wherein the linker sequence according to (iv) comprises the amino acid sequence (GGGGS)n, and wherein n is an integer selected from the group consisting of 1, 2, 3, 4, 5, 6, 7, 8, 9 and 10 and is preferably selected from the group consisting of 2, 3, 4 and 5.
27. The recombinant polypeptide according to any one of the preceding items, wherein said sequence of a human polypeptide domain according to (iii) is at least 95% identical to the amino acid sequence of SEQ ID NO: 5, preferably at least 98% identical to the amino acid sequence of SEQ ID NO: 5 and more preferably identical to the amino acid sequence of SEQ ID NO: 5.
28. The recombinant polypeptide according to any one of the preceding items, wherein said polypeptide is dimeric or multimeric.
29. The recombinant polypeptide according to any one of the preceding items, wherein the polypeptide comprises or consists of all of the components i) to vii)
30. The recombinant polypeptide according to any one of the preceding items, wherein the polypeptide does not comprise components viii) to x).
31. The recombinant polypeptide according to any one of items 1 to 29, wherein the polypeptide comprises or consists of all of the components i) to x).
32. The recombinant polypeptide according to any one of the preceding items, further comprising an N-terminal secretion signal peptide sequence.
33. The recombinant polypeptide according to any one of items 1-31, wherein the recombinant polypeptide consists of an amino acid sequence consisting of the following ((a) and (b)) in an N- to C-terminal order:
   (a) a peptide antigen selected from the group consisting of the amino acid sequences of SEQ ID NO: 2, SEQ ID NO: 22, SEQ ID NO: 23, SEQ ID NO: 24, SEQ ID NO: 25, SEQ ID NO: 26 and SEQ ID NO: 2, and
   (b) the amino acid sequence of SEQ ID NO: 16.
34. The recombinant polypeptide according to any one of the preceding items, wherein the recombinant polypeptide is soluble.
35. A nucleic acid encoding one or more polypeptides according to any one of the preceding items.
36. The nucleic acid according to item 35, wherein the nucleic acid is a vector.
37. A pharmaceutical composition comprising at least one nucleic acid according to items 35 or 36.
38. A pharmaceutical composition or kit comprising at least one recombinant polypeptide according to any one of items 1-34.
39. The pharmaceutical composition or kit according to item 38, wherein the pharmaceutical composition or kit comprises at least two different recombinant polypeptides according to any one of items 1-34, and wherein each of the different polypeptides comprises a different peptide antigen as defined in any one of items 3 to 9.
40. The pharmaceutical composition or kit according to item 38 or 39, wherein the pharmaceutical composition or kit comprises at least the following ((A) to (C)): (A) a recombinant polypeptide wherein said peptide antigen is a peptide antigen of human proinsulin or human insulin; (B) a recombinant polypeptide wherein said peptide antigen is a peptide antigen of human Glutamate decarboxylase 65; (C) a recombinant polypeptide wherein said peptide antigen is a peptide antigen of human Zinc transporter 8; and optionally further comprises (D) a recombinant polypeptide wherein said peptide antigen is a peptide antigen of human islet amyloid polypeptide.
41. The pharmaceutical composition or kit according to any one of items 38 to 40, wherein the pharmaceutical composition or kit comprises at least three different recombinant polypeptides according to any one of items 1-34, wherein said peptide antigen of a first recombinant polypeptide of the at least three different recombinant polypeptides consists of an amino acid sequence of SEQ ID NO: 2, wherein said peptide antigen of a second recombinant polypeptide of the at least three different recombinant polypeptides consists of an amino acid sequence of SEQ ID NO: 22, and wherein said peptide antigen of a third recombinant polypeptide of the at least three different recombinant polypeptides consists of an amino acid sequence of SEQ ID NO: 23.
42. A pharmaceutical composition or kit according to any one of items 37-41, for use in the treatment of type 1 diabetes in a human patient.
43. The pharmaceutical composition or kit for use according to item 42, wherein the treatment is treatment by immunotherapy.
44. The pharmaceutical composition or kit for use according to any one of items 42-43, wherein the treatment is by inducing immunological tolerance against human proinsulin and/or human insulin, human Glutamate decarboxylase 65, human islet amyloid polypeptide and/or human Zinc transporter 8.
45. The pharmaceutical composition or kit for use according to any one of items 42-44, wherein the treatment is for reducing plasma levels of autoantibodies against insulin (insulin autoantibodies IAA), or glutamic acid decarboxylase (GAD-65), or islet antigen-2A (IA-2A), or zinc transporter ZnT8, as assessed by radio-binding assays or non-radioactive electrochemiluminescent antigen-binding assays.
46. The pharmaceutical composition or kit for use according to any one of items 42-45, wherein the human patient is a patient who had plasma autoantibodies against insulin (insulin autoantibodies IAA), or glutamic acid decarboxylase (GAD-65), or islet antigen-2A (IA-2A), or zinc transporter ZnT8 prior to the start of the treatment.
47. A recombinant host cell comprising a nucleic acid or a vector according to item 35 or 36 and expressing the recombinant polypeptide according to any one of items 1-34.
48. A method for obtaining a pharmaceutical composition comprising a polypeptide according to any one of items 1-34, the method comprising the steps of (a) culturing the recombinant host cell of item 47 under conditions allowing expression of the recombinant polypeptide from the nucleic acid molecule, (b) recovering the recombinant polypeptide, (c) purifying the recombinant polypeptide, and (d) formulating the recombinant polypeptide into a pharmaceutical composition.

The pharmaceutical compositions or kits for use of the invention can also be used in a treatment for type 1 diabetes in human patients, wherein the treatment is a co-treatment with a stem cell therapy for regenerating the pancreatic tissue. It is expected that such a co-treatment will be beneficial, since the recombinant polypeptides of the invention will, due to their specific immunosuppressive effect, promote regeneration of the pancreatic tissue by the stem cell therapy.

The pharmaceutical compositions or kits for use of the invention can also be used in a treatment for type 1 diabetes in human patients, wherein the treatment is a co-treatment with a stem cell therapy or a human beta cell regenerative drug therapy for regenerating the pancreatic tissue. Human beta cell regenerative drug therapy for diabetes has been reviewed in P Wang, E Karakose, L Choleva, K Kumar, R J DeVita., A Garcia-Ocaha, A F Stewart Andrew. Human Beta Cell Regenerative Drug Therapy for Diabetes: Past Achievements and Future Challenges. Frontiers in Endocrinology 12, 2021. DOI=10.3389/fendo.2021.671946.

### BRIEF DESCRIPTION OF THE DRAWINGS

FIG. 1: Depiction of a peptide-loaded soluble MHC Ib molecule suitable to achieve therapeutic antigen-specific immunomodulation.

The presented peptide antigens depicted in dotted spheres, the HLA-G alpha1-3 domains are sketched in light-grey, and the beta2microglobulin domain is shown in dark grey. An optional linker connecting the antigenic peptide with the beta2microglobulin molecule is displayed in grey stick style, and an optional disulfide trap is depicted in black spheres. This figure was generated using Pymol and is adapted from structures published in Clements et al., Proc Natl Acad Sci USA. 2005 Mar. 1; 102(9):3360-5 and Hansen et al., Trends Immunol. 2010 October; 31(10):363-9.

FIG. 2: Example for a vector-based construct encoding a single chain MHC Ib molecule suitable for therapeutic peptide-specific immunomodulation.

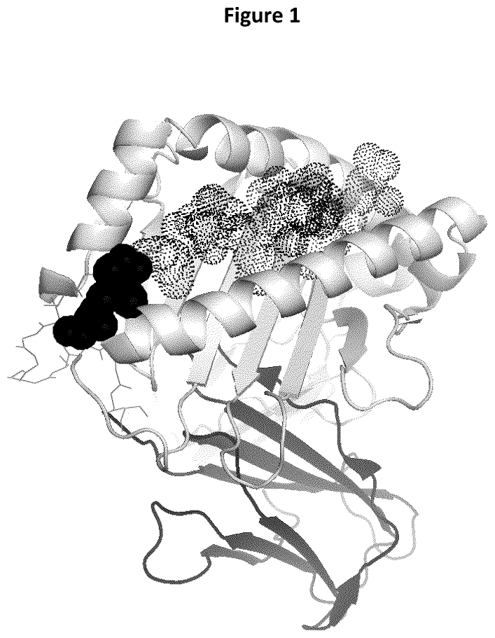

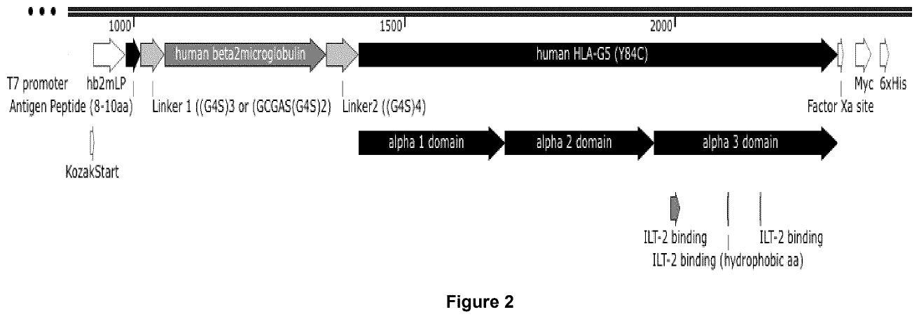

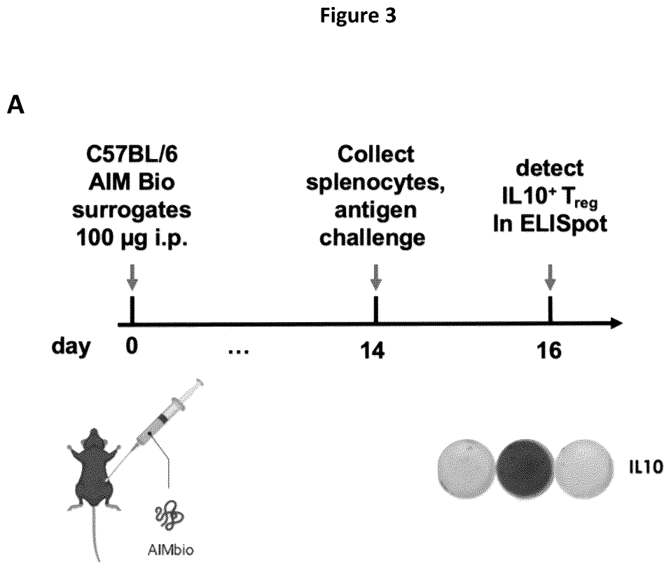

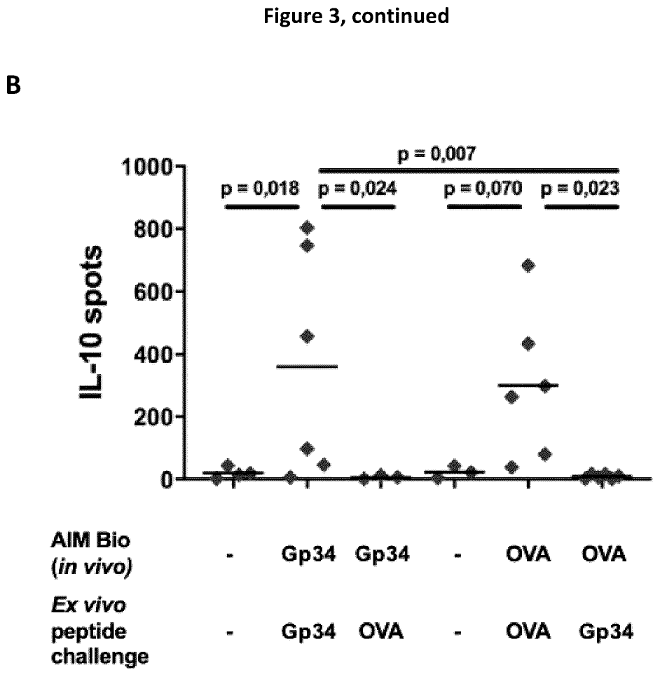

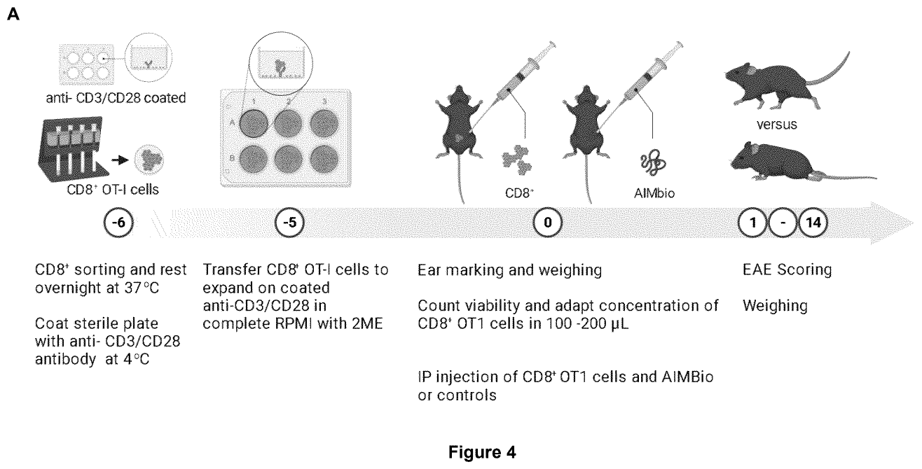

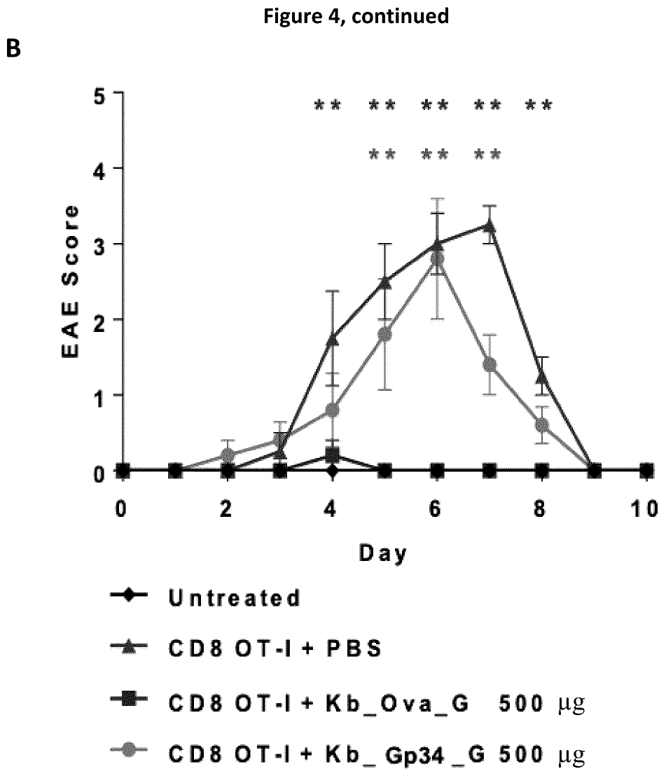

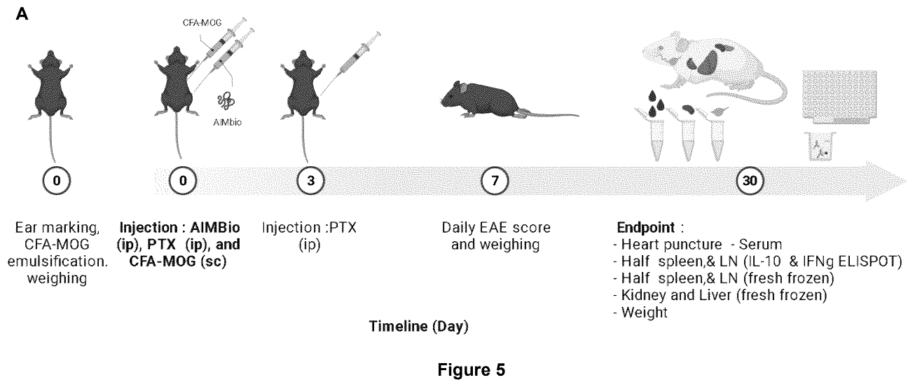

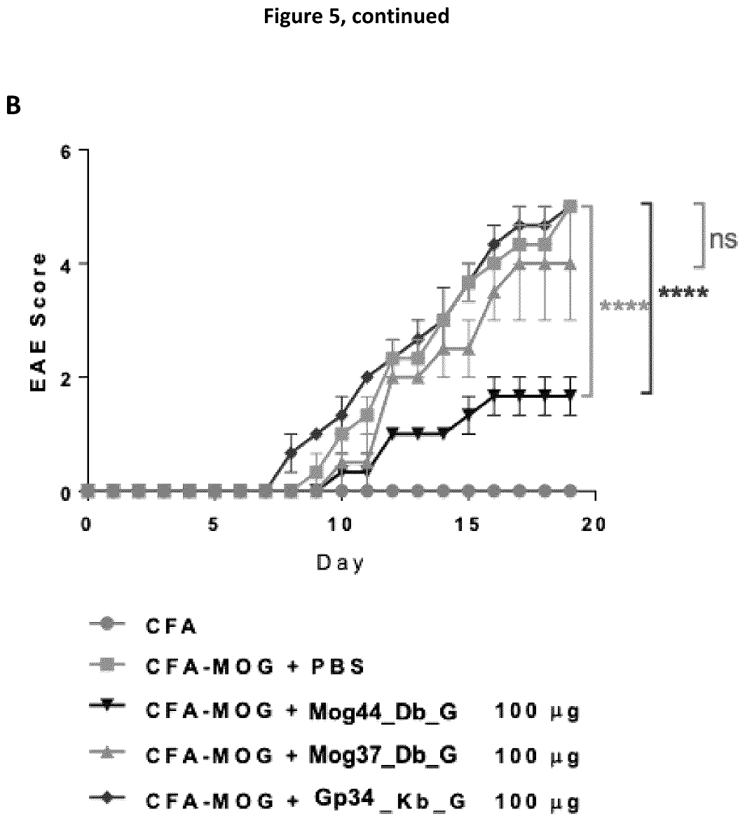

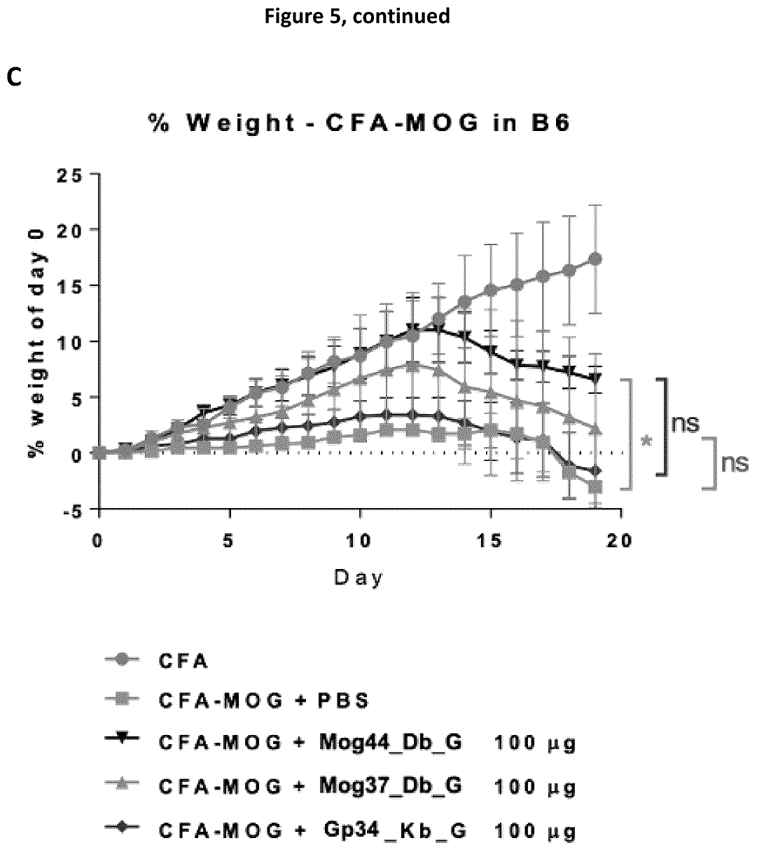

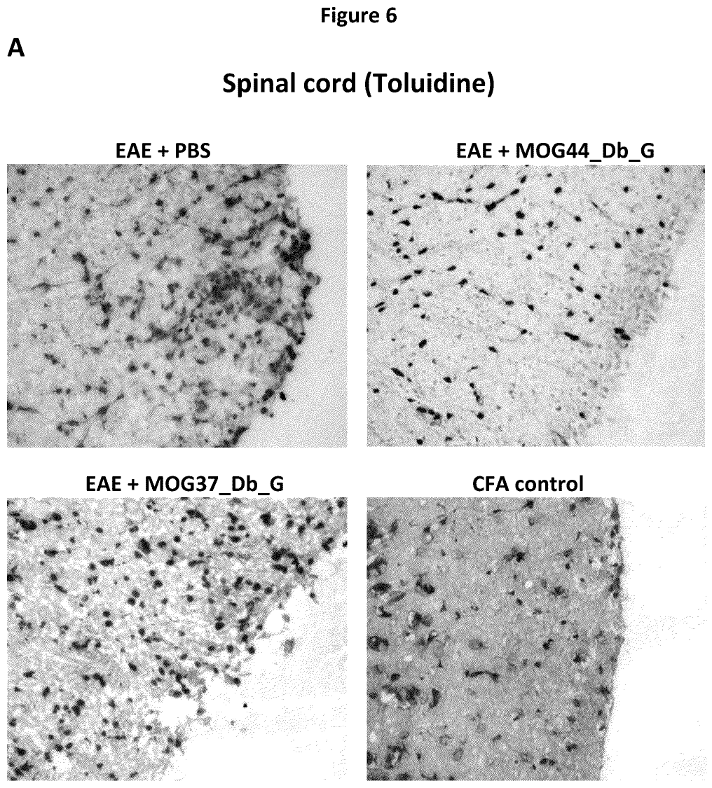

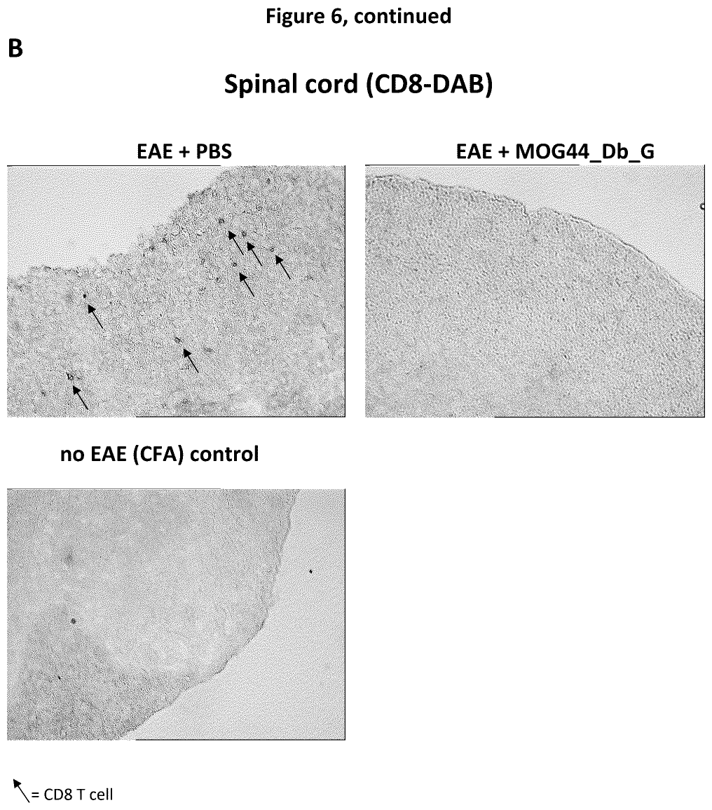

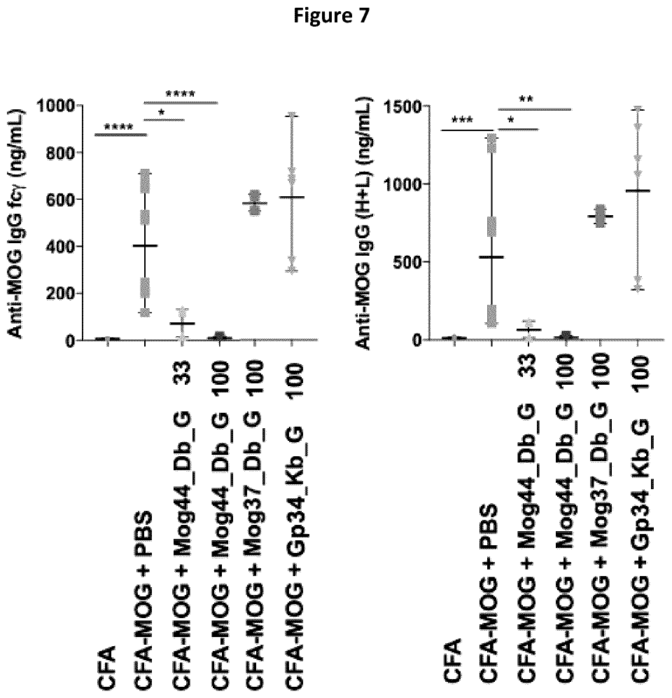

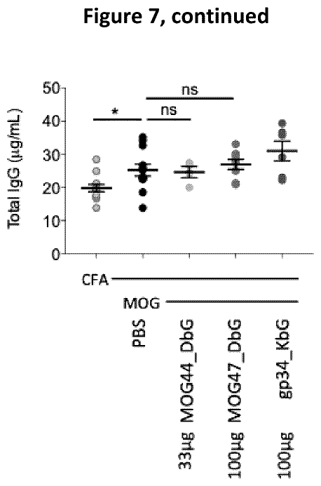

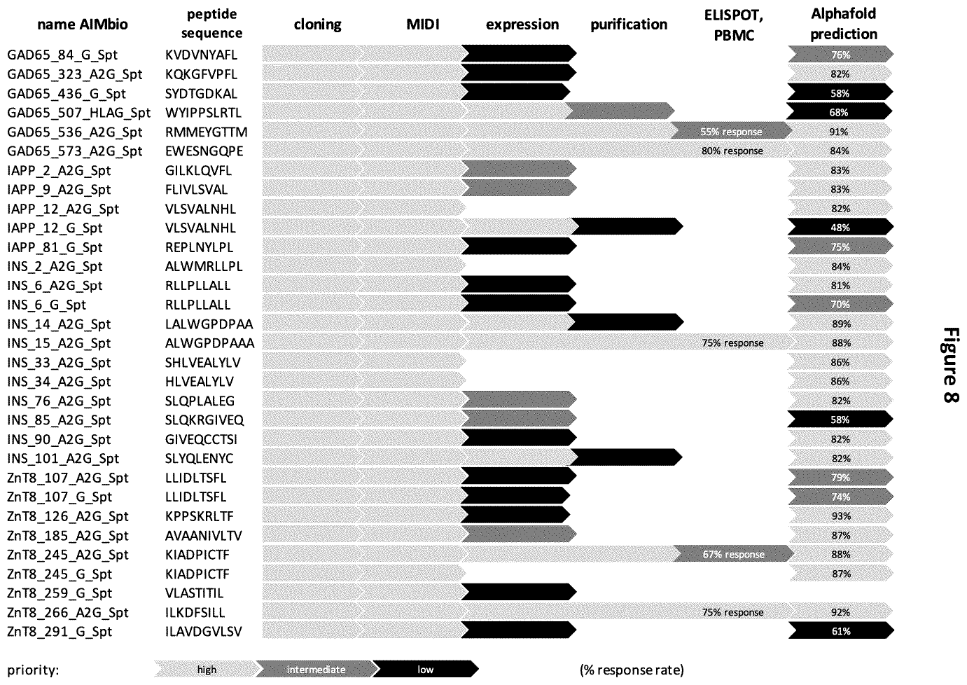

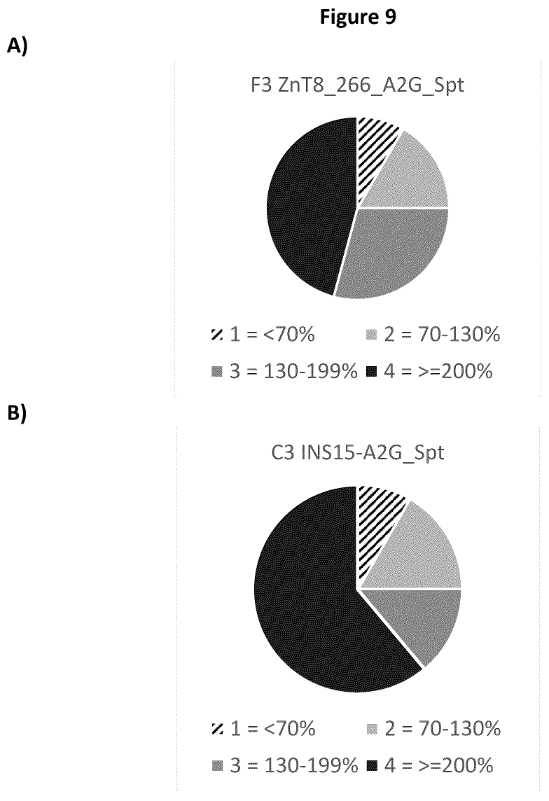

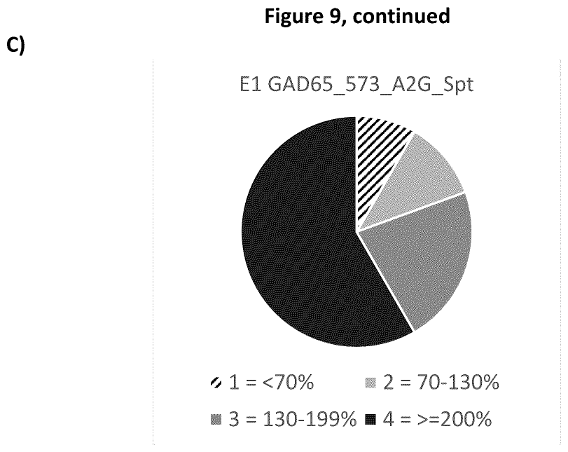

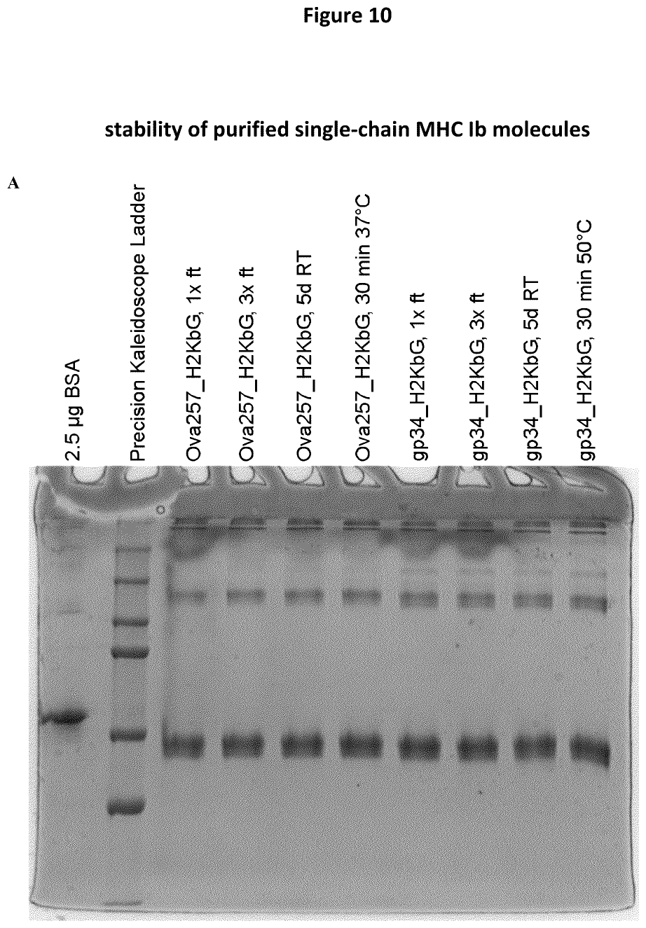

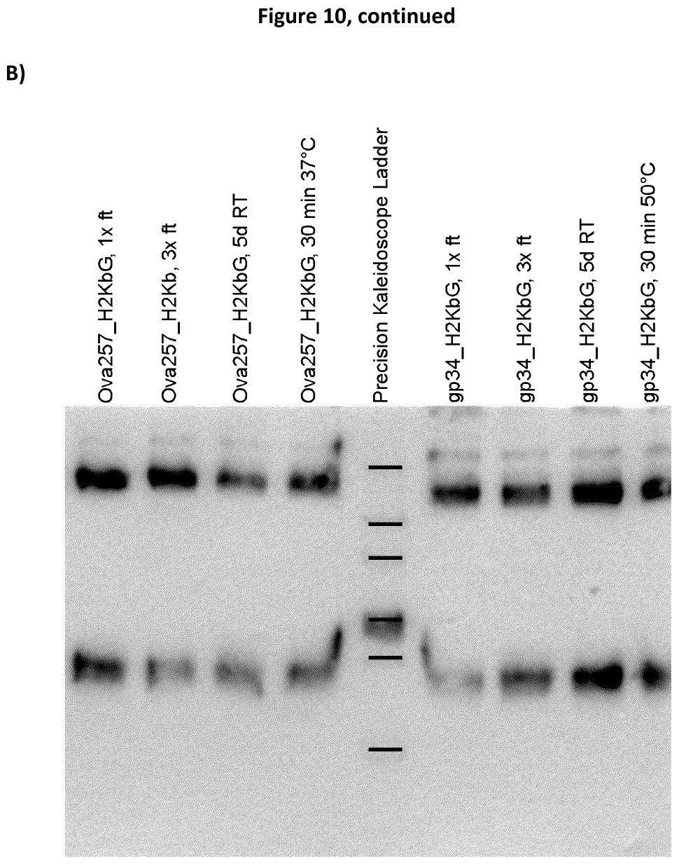

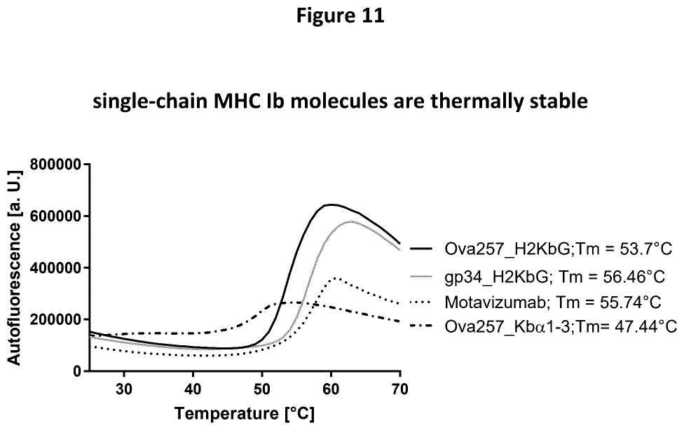

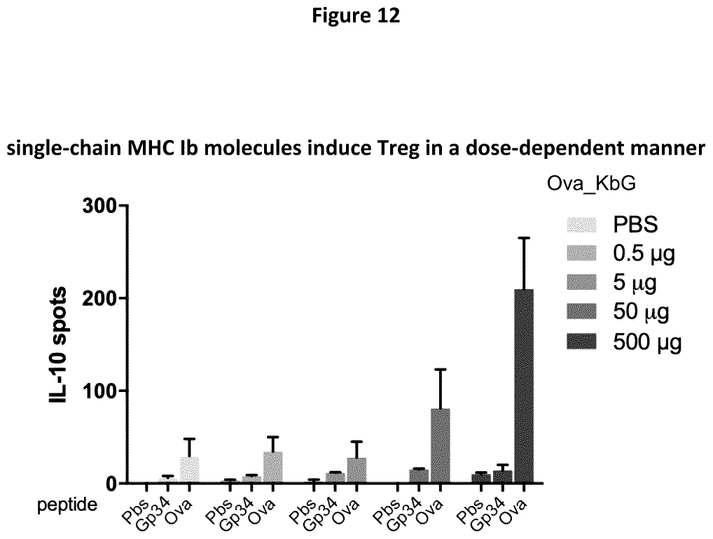

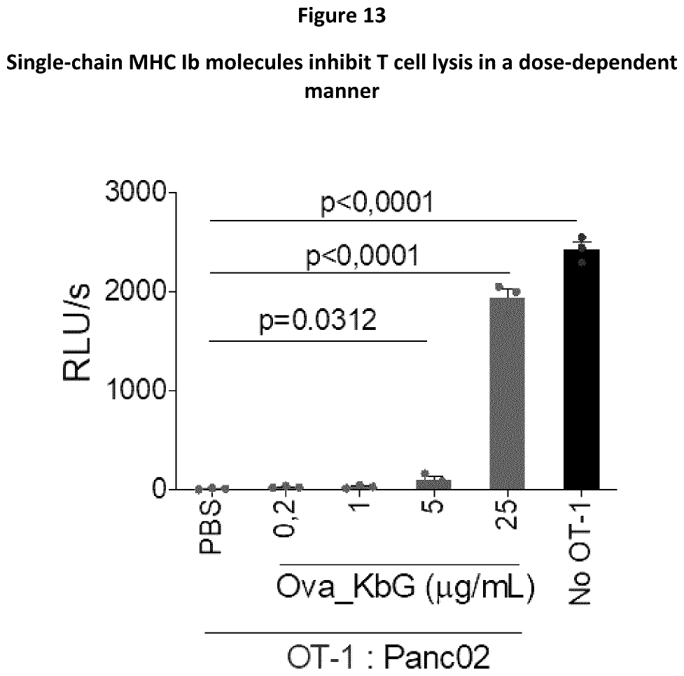

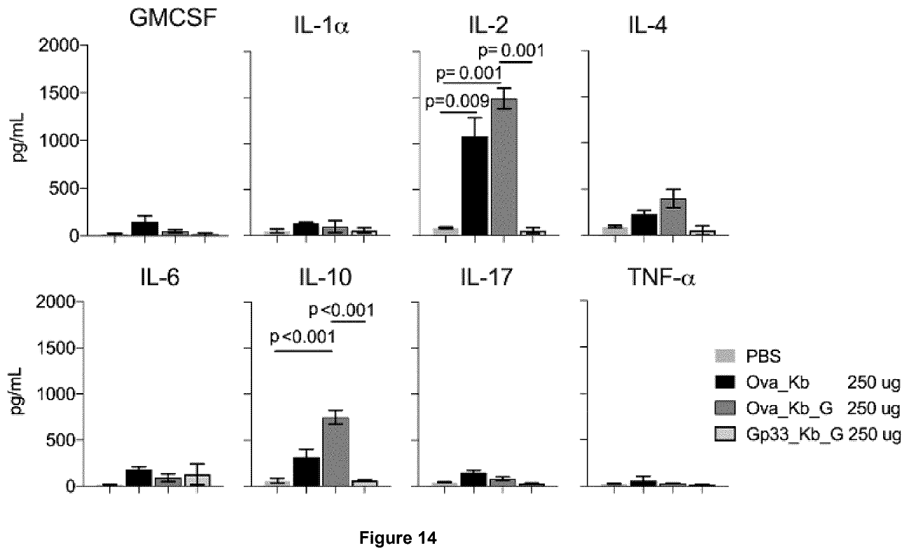

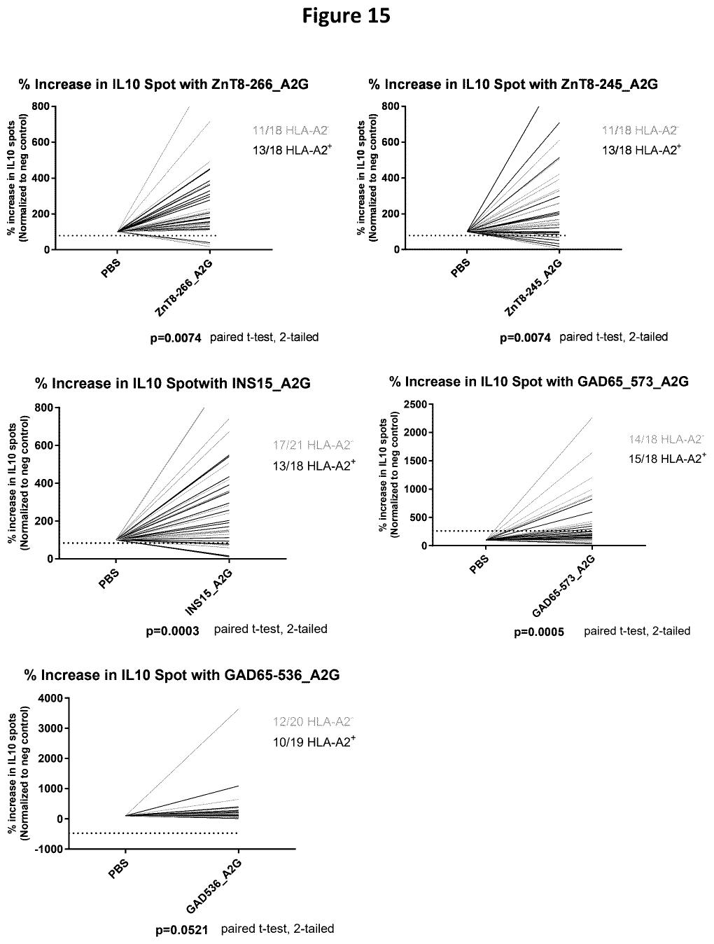

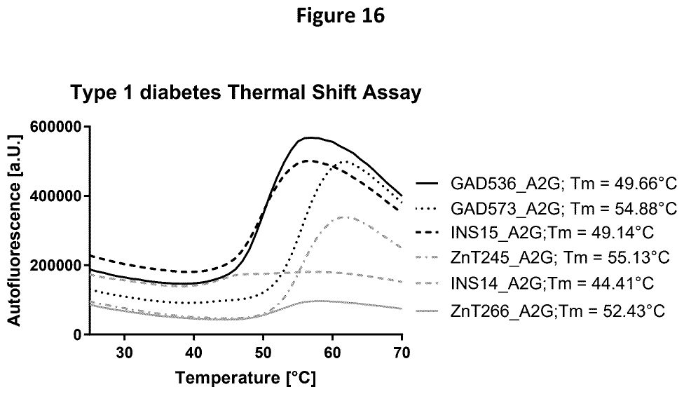

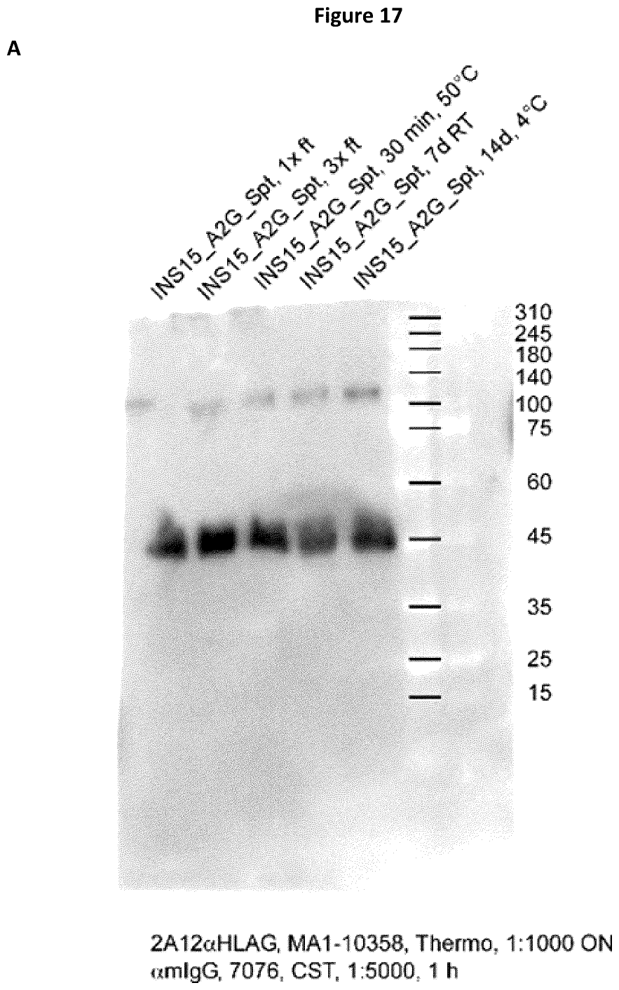

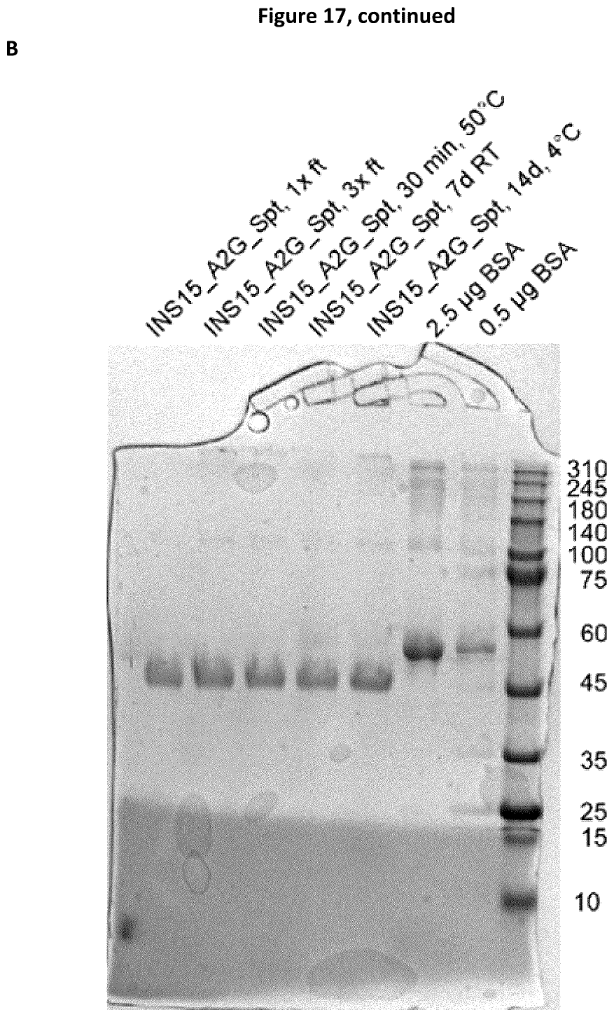

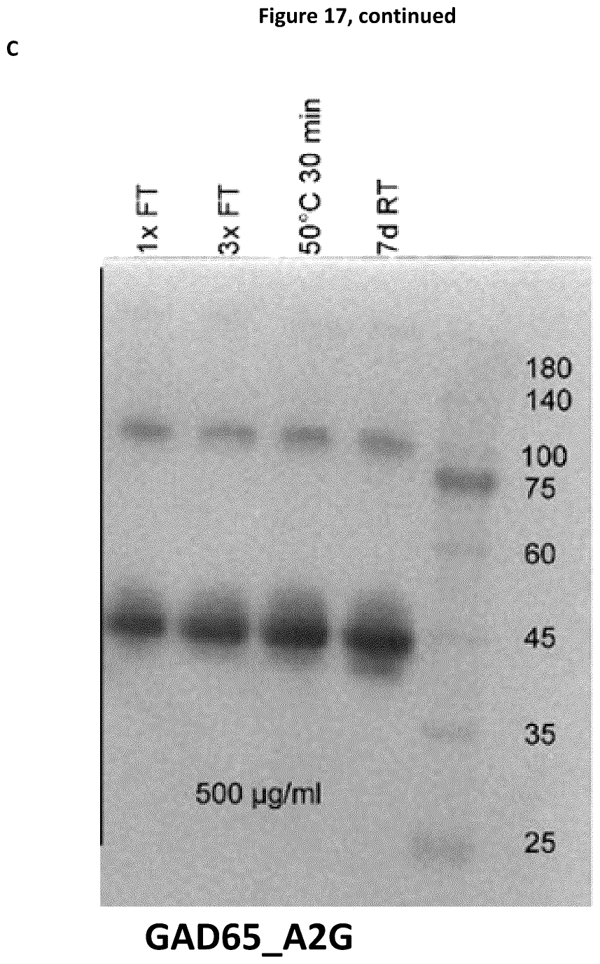

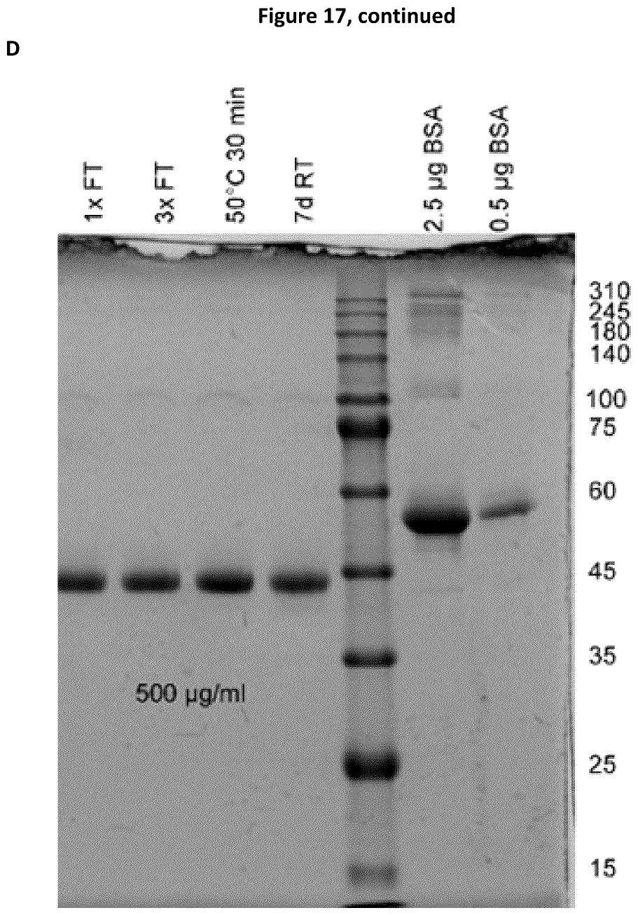

HLA-G1 and HLA-G5 each consist of 3 [alpha] domains (here in black), a non-covalently associated beta 2-microglobulin subunit (here in dark grey) and the antigenic peptide presented on HLA-G (short black arrow).

HLA-G1 further contains a transmembrane domain and a short intracellular chain (not shown here). As shown here, the [alpha]-3 domain is capable of binding to the receptors ILT2 (see Shiroishi et al., Proc Natl Acad Sci USA. 2003 Jul. 22; 100(15):8856-8861) and ILT4 (see Shiroishi et al., Proc Natl Acad Sci USA. 2006 Oct. 31; 103(44):16412-7) on immune cells. Physiologically, these sequences form a non-covalently linked MHC class 1 complex. To simplify purification of the complex MHC Ib molecule, one or more protein tags (such as SpotTag, myc tag and/or His(6×) tag) may be introduced. They may be introduced in such a way as to enable their later optional removal via cleavage using an optional Factor Xa cleavage site. Furthermore, the antigenic peptide, beta 2-microglobulin and MHC Ib [alpha]chain can be linked in order to increase the stability. The vector map was generated using Snapgene Viewer Software.

FIG. 3: Surrogates of recombinant polypeptides of the invention induce IL10 secreting Treg in mice. In this experiment, 100 μg of surrogate molecules consisting of a viral (Gp34) or Ovalbumin (Ova) model peptide antigen, murine H2-K^{b} alpha1 and 2 domains, and human HLA-G alpha 3 domain and beta-2-microglobulin were injected i.p. into 12 week old C57BL/6 mice. After 14 days, mice were sacrificed and splenocytes were rechallenged with 5 μg/ml of either Gp34 or Ova peptide in an 48 h standard murine IL-10 ELIspot assay. A significant increase in regulatory T cells that secreted IL-10 only in response to rechallenge with the peptide towards which tolerance was induced via surrogate molecule injection was detectable.

(A) experimental design; (B) results

FIG. 4: Surrogates of recombinant polypeptides of the invention prevent CD8+ T-cell driven EAE in mice.

In this MS mouse model, the model antigen ovalbumin (OVA) is expressed in oligodendrocytes under the control of the myelin basic protein (MBP) promoter (ODC-OVA). This leads to the presentation of the OVA257-264 peptide on H-2Kb MHC molecules on oligodendrocytes. OT-I mice express a T cell receptor (OT-I) on their CD8+ T cells, which recognizes exactly this peptide-MHC combination. When CD8+ T cells from these mice are transferred into 10 day old ODC-OVA mice, these develop an experimental autoimmune encephalomyelitis (EAE) which resembles in many aspects the pathogenesis and symptomatology of MS (Na et al., Brain, Volume 131, Issue 9, September 2008, Pages 2353-2365). In this experiment, 500 μg of surrogate molecules consisting of a viral (Gp34) or Ovalbumin (Ova) model peptide antigen, murine H2-K^{b} alpha1 and 2 domains, and human HLA-G alpha 3 domain and beta-2-microglobulin or just PBS were injected the same day. EAE was scored according to Bittner et al., J Vis Exp. 2014 Apr. 15; (86):51275.

Only Ovalbumin-tolerance inducing surrogate molecules almost completely prevented EAE symptoms.

(A) experimental design; (B) results

FIG. 5: Some surrogates of recombinant polypeptides of the invention selectively prevent CD4⁺ T cell driven EAE in mice.

In this model, a strong, myelin-specific autoimmune response is triggered by administration of MOG 35-55 peptide in combination with Complete Freund's adjuvant, which activates CD4+ Th17 cells, and pertussis toxin, which makes the blood-brain barrier more permeable (Protocol: Bittner et al., J Vis Exp. 2014 Apr. 15; (86):51275). Here, CD4+ cells as well as antibodies play a crucial role in the development of EAE (Tigno-Aranjuez et al., J Immunol Nov. 1, 2009, 183 (9) 5654-5661). In addition, 100 μg/mouse of surrogate molecules consisting of a viral (Gp34) or two Mog peptide antigens (Mog37 or Mog44), murine H2-D^{b} alpha1 and 2 domains, and human HLA-G alpha 3 domain and beta-2-microglobulin or just PBS were injected the first day.

The Mog44 peptide containing surrogate molecule significantly reduced EAE symptoms and weight loss.

(A) experimental design; (B) EAE score; (C) body weight

FIG. 6: Mog44 surrogates of recombinant polypeptides of the invention prevented inflammation and CD8 T cell infiltration in the spinal cord. (A) Toluidine; (B) CD8-DAB

10 μm fresh frozen sections were stained with commercial Toluidine 1× staining reagent for 1 h at room temperature. A strong infiltration of immune cells was detected in EAE, but prevented by Mog44_Db_G.

10 μm fresh frozen sections were briefly dried at room temperature, ficed with acetone, blocked with 5% BSA 10% normal goat serum in PBS, stained with 1:100 anti-CD8 antibody, secondary antibody coupled to HRP and DAB solution (detailed methods: Karikari et al., Brain Behav Immun. 2022 Jan. 12; 101:194-210)

FIG. 7: Detection of anti-MOG35-55 antibodies in Mog-EAE mice treated with surrogates of recombinant polypeptides of the invention (“AIM Bio”)

Briefly, murine serum was collected from heart puncture after mice were sacrificed. 10 μg/ml Mog35-55 were used for coating over night, wells were blocked using 1% BSA, and anti-Mog35-55 antibodies were detected using the indicated secondary HRP coupled antibodies. FIG. 7, continued, shows that total IgG is not reduces by MOG47_Db_G surrogate molecule treatment in these samples. Easy-Titer™ Human IgG (gamma chain) Assay Kit (Thermo Fisher) was used to quantify total IgG.

FIG. 8: List of the human T1D recombinant polypeptide candidates. Correct protein folding correlates with good or at least acceptable expression. An induction of T reg in PBMC of at least 30% as detected by ELIspot and predicted folding by AlphaFold2 are indicated.

The recombinant polypeptides referred to in the Figure as follows:

| name of recombinant polypeptide | peptide antigen sequence | SEQ ID NO: |
|---|---|---|
| T1D-01 GAD65_84_G_Spt | KVDVNYAFL | SEQ ID NO: 38 |
| T1D-02 GAD65_323_A2G_Spt | KQKGFVPFL | SEQ ID NO: 39 |
| T1D-03 GAD65_436_G_Spt | SYDTGDKAL | SEQ ID NO: 40 |
| T1D-04 GAD65_507_HLAG_Spt | WYIPPSLRTL | SEQ ID NO: 26 |
| T1D-05 GAD65_536_A2G_Spt | RMMEYGTTM | SEQ ID NO: 27 |
| T1D-06 GAD65_573_A2G_Spt | EWESNGQPE | SEQ ID NO: 23 |
| T1D-07 IAPP_2_A2G_Spt | GILKLQVFL | SEQ ID NO: 41 |
| T1D-08 IAPP_9_A2G_Spt | FLIVLSVAL | SEQ ID NO: 42 |
| T1D-09 IAPP_12_A2G_Spt | VLSVALNHL | SEQ ID NO: 43 |
| T1D-10 IAPP_12_G_Spt | VLSVALNHL | SEQ ID NO: 43 |
| T1D-11 IAPP_81_G_Spt | REPLNYLPL | SEQ ID NO: 44 |
| T1D-12 INS_2_A2G_Spt | ALWMRLLPL | SEQ ID NO: 45 |
| T1D-13 INS_6_A2G_Spt | RLLPLLALL | SEQ ID NO: 46 |
| T1D-14 INS_6_G_Spt | RLLPLLALL | SEQ ID NO: 46 |
| T1D-15 INS_14_A2G_Spt | LALWGPDPAA | SEQ ID NO: 25 |
| T1D-16 INS_15_A2G_Spt | ALWGPDPAAA | SEQ ID NO: 2 |
| T1D-17 INS_33_A2G_Spt | SHLVEALYLV | SEQ ID NO: 47 |
| T1D-18 INS_34_A2G_Spt | HLVEALYLV | SEQ ID NO: 48 |
| T1D-19 INS_76_A2G_Spt | SLQPLALEG | SEQ ID NO: 49 |
| T1D-20 INS_85_A2G_Spt | SLQKRGIVEQ | SEQ ID NO: 50 |
| T1D-21 INS_90_A2G_Spt | GIVEQCCTSI | SEQ ID NO: 51 |
| T1D-22 INS_101_A2G_Spt | SLYQLENYC | SEQ ID NO: 52 |
| T1D-23 ZnT8_107_A2G_Spt | LLIDLTSFL | SEQ ID NO: 53 |
| T1D-24 ZnT8_107_G_Spt | LLIDLTSFL | SEQ ID NO: 53 |
| T1D-25 ZnT8_126_A2G_Spt | KPPSKRLTF | SEQ ID NO: 54 |
| T1D-26 ZnT8_185_A2G_Spt | AVAANIVLTV | SEQ ID NO: 55 |
| T1D-27 ZnT8_245_A2G_Spt | KIADPICTF | SEQ ID NO: 24 |
| T1D-28 ZnT8_245_G_Spt | KIADPICTF | SEQ ID NO: 24 |
| T1D-29 ZnT8_259_G_Spt | VLASTITIL | SEQ ID NO: 56 |
| T1D-30 ZnT8_266_A2G_Spt | ILKDFSILL | SEQ ID NO: 22 |
| T1D-31 ZnT8_291_G_Spt | ILAVDGVLSV | SEQ ID NO: 57 |

FIG. 9: Upregulation of CD8 Treg in healthy blood donors by recombinant polypeptides of the invention. The figure shows upregulation of CD8⁺ Treg cells by recombinant polypeptides containing the Zinc transporter 8 peptide antigen ILKDFSILL (A), the insulin peptide antigen ALWGPDPAAA (B) and the Glutamate decarboxylase 65 peptide antigen EWESNGQPE (C), respectively.

FIG. 10: Purified single-chain MHC Ib molecules are stable monomers or dimers. After purification of the single chain MHC Ib molecules for FIGS. 3 and 4, their stability was analysed after 1 and 3 freeze-thawing cycles, storage for 5 days at room temperature and heating up to a temperature of 50° C. for 30 min. For this, A) a Coomassie gel staining of a 12% polyacrylamide gel using 2 μg AIM Bio and B) an aHLA-G Western blot using the 2A12aHLA-G antibody (1:1000) blot using 1 μg protein was performed under non-reducing conditions. Both monomers and dimers are detectable.

FIG. 11: Single-chain MHC Ib molecules are thermally stable. For the Thermal Shift Assay (TSA), 3 μg of the respective single chain MHC Ib molecule or Motavizumab as control molecule were diluted with PBS and 5×SYPRO Orange dye (stock 5000×, final concentration: 5×) to a volume of 25 μl. A melting curve program was set up on a StepOnePlus Instrument using the StepOnePlus Software 2.3. The start temperature was 25° C. for one minute followed by a temperature increase of 1° C. per minute to a final temperature of 95° C. for 2 min, thereby measuring the autofluorescence as arbitrary unit. Data were exported and graphs were drawn in Prism V7.04. For determination of the melting temperature (Tm), the Boltzman sigmoidal function was used.

FIG. 12: Single-chain MHC Ib molecules induce Treg in a dose-dependent manner. OT-I mice were injected i.p. with indicated amounts of single-chain H2_K^{b} alpha1+2 and HLA-G alpha3 domain constructs with human beta-2-microglobulin and the indicated peptide or carrier (PBS). Ova is the cognate peptide for the OT-I TCR in these mice, Gp34 is an irrelevant, virus derived control peptide. After 14 days, mice were sacrificed and splenocytes tested for IL10 secreting cells in a recall mouse IL-10 ELISpot (200,000 cells per well, MabTech mouse IL-10 ELISpot kit, 5 μg/ml of the indicated peptide or only PBS were added, 48 h). A clear induction of IL-10 secreting cells reactive to Ova peptide was observed when 50 and 500 μg mouse adapted Ova_KbG were injected.

FIG. 13: Single-chain MHC Ib molecules inhibit T cell lysis in a dose-dependent manner. OT1/BL6 Mice were sacrificed and splenocytes were collected and washed once in RPMI 5% FCS. Red blood cells were removed with 2 ml 1× sterile RBC lysis buffer for 3 min. Cells were cultured in high density culture (10 mio cells/ml) for 72 h in RPMI 10% FCS medium with GMCSF 20 ng/mL, IL-2 20 ng/ml and IL-4 10 ng/ml and increasing doses of Ova_KbG. Cells are then scraped from the plates, CD8+ cells are then purified via magnetic beads.

Sterile 96-well white plates were used. Luciferase expressing Panc02 target cells were loaded with 20 μg/ml Ova peptide (SIINFEKL) for 60 min at 37° C. with 500 rpm shaking. CD8+ effector T cells were added in a 50:1 ratio, as well as luciferin. Luminescence was measured after 0 h, 24 h, 48 h.

FIG. 14: Single-chain MHC Ib molecules induce expression of IL-10 in EAE-ODC Ova mice. Serum cytokines from EAE-ODC Ova mice were measured with Th1/Th2 10plex Flowcytomix Kit (eBioscience) according to the manufacturer's instruction. The kit was used for the simultaneous detection of mouse granulocyte-macrophage colony-stimulating factor (GMCSF), interleukin 1 alpha (IL-1a), interleukin-2 (IL-2), interleukin-4 (IL-4), interleukin-6 (IL-6), interleukin-10 (IL-10), t interleukin-17 (IL-17), and tumor necrosis factor alpha (TNF-α) in a single sample. Beads coated with eight specific capture antibodies were mixed. Subsequently, 25 μL of the mixed captured beads, 25 μL of the unknown serum sample or standard dilutions, and 25 μL of phycoerythrin (PE) detection reagent were added consecutively to each well in 96-V bottom well plates and incubated for 2 h at room temperature in the dark. The samples were washed with 1 mL of wash buffer for 5 min and centrifuged. The bead pellet was resuspended in 200 μL buffer after discarding the supernatant. Samples were measured on the Attune™ NxT Flow Cytometer and analyzed Attune Cytometric Software (Thermo Fisher Scientific).

FIG. 15: Increase in IL-10 secreting T cells in response to treatment with the indicated single chain MHC Ib molecule (recombinant polypeptide of the invention).

% increase in IL-10 secreting T cells in response to treatment with the indicated single chain MHC Ib molecule is shown. Black lines indicate an HLA-A2 positive, grey a negative donor. Response a significant increase of Treg is observed bot in HLA-A2 positive and negative donors (response rate indicated in legend)

FIG. 16: Thermal Shift Assay

For the Thermal Shift Assay (TSA), 3 μg of the respective single chain MHC Ib molecule were diluted with PBS and 5×SYPRO Orange dye (stock 5000×, final concentration: 5×) to a volume of 25 μl. A melting curve program was set up on a StepOnePlus Instrument using the StepOnePlus Software 2.3. The start temperature was 25° C. for one minute followed by a temperature increase of 1° C. per minute to a final temperature of 95° C. for 2 min, thereby measuring the autofluorescence as arbitrary unit. Data were exported and graphs were drawn in Prism V7.04. For determination of the melting temperature (Tm), the Boltzman sigmoidal function was used. The high melting temperatures indicate good protein stability for therapeutic use.

FIG. 17: Thermal Stability Assay

A, C: Western Blots of the indicated recombinant polypeptides. B, D: Coomassie Gels of the indicated recombinant polypeptides (using the same methods).

The data indicate that T1D single chain MHC Ib molecules (recombinant polypeptides of the invention) can be purified and stored and are resistant to freeze-thaw cycles

### DETAILED DESCRIPTION OF THE INVENTION

### Definitions and General Techniques

Unless otherwise defined below, the terms used in the present invention shall be understood in accordance with their common meaning known to the person skilled in the art. All publications, patents and patent applications cited herein are hereby incorporated by reference in their entirety for all purposes. Publications referred to herein may be cited either by specifying the full literature reference in the text.

All proteins in accordance with the invention, including the recombinant polypeptides of the invention, can be obtained by methods known in the art. Such methods include methods for the production of recombinant polypeptides. The recombinant polypeptides of the invention can be expressed in recombinant host cells according to the invention. Recombinant host cells of the invention are preferably mammalian cells such as CHO and HEK cells.

It will be understood that the recombinant polypeptides of the invention are meant to optionally include a secretion signal peptide sequence. Similarly, the recombinant polypeptides of the invention are meant to also optionally include affinity tags, e.g. in order to facilitate purification, and optional protease cleavage sites between the tag and the polypeptide, e.g. in order to facilitate removal of the tags by protease cleavage.

It is also understood that any reference to amino acid sequences referred to herein is meant to encompass not only the unmodified amino acid sequence but also typical posttranslational modifications of these amino acid sequences (e.g., glycosylation or deamidation of amino acids, the clipping of particular amino acids or other posttranslational modifications) occurring in cellular expression systems known in the art, including mammalian cells such as CHO and HEK cells.

Likewise, it will be understood that the recombinant polypeptides of the invention are meant to optionally include the respective pro-peptides.

It will also be understood that the recombinant polypeptides of the invention can be in form of their soluble or their membrane-bound form. As used herein, the term “soluble” means that the recombinant polypeptide is soluble under the following reference conditions: 5 μg/ml to 5 mg/ml in PBS, optionally with 0.1% human serum, or optionally in 50% glycerol. Whether a recombinant polypeptide is “soluble” under these conditions can be determined by methods known in the art, e.g., by measuring the turbidity of the recombinant polypeptide under the above-indicated reference conditions. As used herein, soluble means that at least 95% of the recombinant polypeptide is determined to be soluble under these reference conditions. Single chain MHC molecules can be stored, for instance, in PBS at −80° C. (with or without 0.1% human albumin as carrier, depending on the protein concentration) or in 50% glycerol at −20° C.

According to the invention, MHC molecules are preferably human MHC molecules.

The recombinant polypeptides of the invention are preferably isolated recombinant polypeptides.

It will be understood how a recombinant polypeptide capable of binding and presenting an peptide antigen according to the invention can be prepared. For example, peptide antigen-binding domains such as [alpha]1 and [alpha]2 domains are well-known, and modifications of these domains can be made. The capability of a peptide antigen to bind to the polypeptides and MHC molecules according to the invention can be determined by techniques known in the art, including but not limited to explorative methods such as MHC peptide elution followed by Mass spectrometry and bio-informatic prediction in silico, and confirmative methods such as MHC peptide multimere binding methods and stimulation assays.

In accordance with the invention, the recombinant polypeptides, pharmaceutical compositions and kits of the invention are preferably suitable for use in a human patient.

In accordance with the invention, the recombinant polypeptides, pharmaceutical compositions and kits of the invention are preferably suitable for use in the treatment of type 1 diabetes in a human patient.

In accordance with the invention, the recombinant polypeptides, pharmaceutical compositions and kits of the invention are preferably suitable for inducing immunological tolerance against human proinsulin or human insulin, human Glutamate decarboxylase 65, human islet amyloid polypeptide or human Zinc transporter 8, e.g., in a human patient.

It is understood that in accordance with the invention, the recombinant polypeptides, pharmaceutical compositions and kits of the invention are stable.

It will be understood that in connection with the peptide antigens used in accordance with the invention, any lengths of these peptide antigens referred to herein (e.g. “7 to 11 amino acids in length”) are meant to refer to the length of the peptide antigens themselves. Thus, the lengths of peptide antigens referred to herein do not include the length conferred by additional amino acids which are not part of the peptide antigens such as additional amino acids from possible linker sequences etc.

In accordance with the present invention, each occurrence of the term “comprising” may optionally be substituted with the term “consisting of”.

### METHODS AND TECHNIQUES

Generally, unless otherwise defined herein, the methods used in the present invention (e.g. cloning methods or methods relating to antibodies) are performed in accordance with procedures known in the art, e.g. the procedures described in Sambrook et al. (“Molecular Cloning: A Laboratory Manual.”, 2^{nd} Ed., Cold Spring Harbor Laboratory Press, Cold Spring Harbor, New York 1989), Ausubel et al. (“Current Protocols in Molecular Biology.” Greene Publishing Associates and Wiley Interscience; New York 1992), and Harlow and Lane (“Antibodies: A Laboratory Manual” Cold Spring Harbor Laboratory Press, Cold Spring Harbor, New York 1988), all of which are incorporated herein by reference.

Protein-protein binding, such as binding of antibodies to their respective target proteins, can be assessed by methods known in the art. Protein-protein binding is preferably assessed by surface plasmon resonance spectroscopy measurements.

For instance, binding of MHC class Ib molecules or recombinant polypeptides according to the invention to their receptors, including ILT2 and ILT4, is preferably assessed by surface plasmon resonance spectroscopy measurements. More preferably, binding of MHC class Ib molecules or recombinant polypeptides according to the invention to their receptors is assessed by surface plasmon resonance measurements at 25° C. Appropriate conditions for such surface plasmon resonance measurements have been described by Shiroishi et al., Proc Natl Acad Sci USA. 2003 Jul. 22; 100(15):8856-8861.

Sequence Alignments of sequences according to the invention are performed by using the BLAST algorithm (see Altschul et al. (1990) “Basic local alignment search tool.” Journal of Molecular Biology 215. p. 403-410.; Altschul et al.: (1997) Gapped BLAST and PSI-BLAST: a new generation of protein database search programs. Nucleic Acids Res. 25:3389-3402.). Appropriate parameters for sequence alignments of short peptides by the BLAST algorithm, which are suitable for peptide antigens in accordance with the invention, are known in the art. Most software tools using the BLAST algorithm automatically adjust the parameters for sequence alignments for a short input sequence. In one embodiment, the following parameters are used: Max target sequences 10; Word size 3; BLOSUM 62 matrix; gap costs: existence 11, extension 1; conditional compositional score matrix adjustment. Thus, when used in connection with sequences, terms such as “identity” or “identical” preferably refer to the identity value obtained by using the BLAST algorithm.

### Preparation of Pharmaceutical Compositions of the Invention

Pharmaceutical compositions of the present invention are prepared in accordance with known standards for the preparation of pharmaceutical compositions.

For instance, the pharmaceutical compositions are prepared in a way that they can be stored and administered appropriately. The pharmaceutical compositions of the invention may therefore comprise pharmaceutically acceptable components such as carriers, excipients and/or stabilizers.

Such pharmaceutically acceptable components are not toxic in the amounts used when administering the pharmaceutical composition to a human patient. The pharmaceutical acceptable components added to the pharmaceutical compositions may depend on the chemical nature of the active ingredients present in the composition, the particular intended use of the pharmaceutical compositions and the route of administration. In general, the pharmaceutically acceptable components used in connection with the present invention are used in accordance with knowledge available in the art, e.g. from Remington's Pharmaceutical Sciences, Ed.

AR Gennaro, 20th edition, 2000, Williams & Wilkins, PA, USA. Pharmaceutical compositions comprising the nucleic acids of the invention (e.g., RNAs) may also be formulated in accordance with knowledge available in the art, e.g. using liposomal formulations targeting dendritic cells.

Peptide Antigens in Accordance with the Invention

The peptide antigens which can be used in accordance with the invention, including the peptide antigens as defined above, are not particularly limited other than by their ability to be presented on MHC molecules. It is understood that a “peptide antigen presented by said recombinant polypeptide” as referred to in relation to the invention is a peptide antigen that is presented by said recombinant polypeptide to human T cells if such T cells are present.

Peptides which are able to be presented on MHC molecules can be generated as known in the art (see, for instance, Rammensee, Bachmann, Emmerich, Bachor, Stevanović. SYFPEITHI: database for MHC ligands and peptide motifs. Immunogenetics. 1999 November; 50(3-4):213-9; Pearson et al. MHC class I-associated peptides derive from selective regions of the human genome. J Clin Invest. 2016 Dec. 1; 126(12):4690-4701; and Rock, Reits, Neefjes. Present Yourself! By MHC Class I and MHC Class II Molecules. Trends Immunol. 2016 November; 37(11):724-737).

Peptide antigens are generally known in the art. Generally, the peptide antigens in accordance with the invention are capable of binding to MHC class I proteins. It will be understood by a person skilled in the art that for each MHC class Ib molecule or polypeptide capable of presenting peptides in accordance with the invention, peptide antigens which are capable of binding to said MHC class Ib molecule or recombinant polypeptide will preferably be used. These peptide antigens can be selected based on methods known in the art.

Binding of peptide antigens to MHC class Ib molecules or to polypeptides capable of peptide antigen binding in accordance with the invention can be assessed by methods known in the art, e.g. the methods of:
Rammensee, Bachmann, Emmerich, Bachor, Stevanović. SYFPEITHI: database for MHC ligands and peptide motifs. Immunogenetics. 1999 November; 50(3-4):213-9;
Pearson et al. MHC class I-associated peptides derive from selective regions of the human genome. J Clin Invest. 2016 Dec. 1; 126(12):4690-4701; and
Rock, Reits, Neefjes. Present Yourself! By MHC Class I and MHC Class II Molecules. Trends Immunol. 2016 November; 37(11):724-737.

Such methods include experimental methods and methods for the prediction of peptide antigen binding. Anchor residues which serve to anchor the peptide antigen on the MHC class I molecule and to ensure binding of the peptide antigen to the MHC class I molecule are known in the art.

In a preferred embodiment in accordance with all embodiments of the invention, the peptide antigen used in accordance with the invention contain any of the anchor or preferred amino acid residues in the positions as predicted for MHC class I molecules.

Such predictions can preferably be made in as described in any one of the following publications:
Rammensee et al, SYFPEITHI: database for MHC ligands and peptide motifs. Immunogenetics (1999) 50: 213-219
Nielsen et al, Protein Sci (2003) 12:1007-1017
Neefjes et al. Nat Rev Immunol. 2011 Nov. 11; 11(12):823-36
Diehl et al. Curr Biol. 1996 Mar. 1; 6(3):305-14,
Lee et al. Immunity. 1995 November; 3(5):591-600.
Desai & Kulkarni-Kale, T-cell epitope prediction methods: an overview. Methods Mol Biol. 2014; 1184:333-64.
Jumper et al. Highly accurate protein structure prediction with AlphaFold. Nature 2021; 596:583-589

In the invention, the peptide antigen is from human proinsulin or human insulin, human Glutamate decarboxylase 65, human islet amyloid polypeptide or human Zinc transporter 8.

It is understood that the non-anchor amino acid residues of the peptide antigen of the invention may or may not contain conservative substitutions, preferably not more than two conservative substitutions, more preferably one conservative substitution with respect to the corresponding amino acid sequence of a peptide antigen from human proinsulin or human insulin, human Glutamate decarboxylase 65, human islet amyloid polypeptide or human Zinc transporter 8.

Peptide antigens of the invention preferably consist of naturally occurring amino acids. However, non-naturally occurring amino acids such as modified amino acids can also be used. For instance, in one embodiment, a peptide antigen of the invention encompasses the peptidomimetic of the indicated peptide antigen amino acid sequence of human proinsulin or human insulin, human Glutamate decarboxylase 65, human islet amyloid polypeptide or human Zinc transporter 8.

Methods for the synthesis of peptide antigens, including peptide antigens in accordance with the invention, are well known in the art.

### Sequences

Preferred amino acid sequences referred to in the present application can be independently selected from the following sequences. The sequences are represented in an N-terminal to C-terminal order; and they are represented in the one-letter amino acid code.

Exemplary sequences which are part of the recombinant polypeptides of the invention: Optional leader Peptide (absent from the recombinant polypeptide due to processing during cellular expression): e.g. MSRSVALAVLALLSLSGLEA (SEQ ID NO: 1)
Peptide antigen: an MHC class I peptide corresponding to MHC class I [alpha] 1&2 domains, e.g.

(SEQ ID NO: 2)
ALWGPDPAAA
First linker: For instance
(SEQ ID NO: 3)
GGGGSGGGGSGGGGS
or
(SEQ ID NO: 4)
GCGASGGGGSGGGGS

beta 2 Microglobulin, for instance:

(SEQ ID NO: 5, human beta 2 Microglobulin)
IQRTPKIQVYSRHPAENGKSNFLNCYVSGFHPSDIEVDLLKNGERIEKV
EHSDLSFSKDWSFYLLYYTEFTPTEKDEYACRVNHVTLSQPKIVKWDRD
M

Second Linker, for instance:

(SEQ ID NO: 6)
GGGGSGGGGSGGGGSGGGGS

[Alpha] 1 & 2 domain derived either from human HLA-G or from any other MHC class I [alpha]1&2 domain suitable to present the selected antigenic peptide, Y84 may be C in DT variant
e.g. [Alpha]1 & 2 domain derived from human HLA-G: E.g.

(SEQ ID NO: 7)
GSHSMRYFSAAVSRPGRGEPRFIAMGYVDDTQFVRFDSDSACPRMEPRA
PWVEQEGPEYWEEETRNTKAHAQTDRMNLQTLRGCYNQSEASSHTLQWM
IGCDLGSDGRLLRGYEQYAYDGKDYLALNEDLRSWTAADTAAQISKRKC
EAANVAEQRRAYLEGTCVEWLHRYLENGKEMLQRA

Or: Human HLA-A2 [alpha]1 &2 domain: E.g.

(SEQ ID NO: 8)
GSHSMRYFFTSVSRPGRGEPRFIAVGYVDDTQFVRFDSDAASQRMEPRA
PWIEQEGPEYWDGETRKVKAHSQTHRVDLGTLRGCYNQSEAGSHTVQRM
YGCDVGSDWRFLRGYHQYAYDGKDYIALKEDLRSWTAADMAAQTTKHKW
EAAHVAEQLRAYLEGTCVEWLRRYLENGKETLQRT

Human HLA-G [alpha]3 domain (or any MHC Ib [alpha]3 domain, such as HLA-F, which also interacts with ILT2 and ILT4 receptors), for instance:

(SEQ ID NO: 9; sequence of HLA-G [alpha]3)
DPPKTHVTHHPVFDYEATLRCWALGFYPAEIILTWQRDGEDQTQDVELV
ETRPAGDGTFQKWAAVVVPSGEEQRYTCHVQHEGLPEPLMLRWSKEGDG
GIMSVRESRSLSEDL.

Note that the following underlined amino acids of this sequence are relevant for ILT2 or ILT4 receptor interaction:

DPPKTHVTHHPVFDYEATLRCWALGFYPAEIILTWQRDGED
QTQDVELVETRPAGDGTFQKWAAVVVPSGEEQRYTCHVQHE
GLPEPLMLRWSKEGDGGIMSVRESRSLSEDL

Alternatively, a shorter form of a human HLA-G [alpha]3 domain may be used which lacks the optional C-terminal amino acid sequence from intron 4 (SKEGDGGIMSVRESRSLSEDL; SEQ ID NO: 20), i.e.:

(SEQ ID NO: 21)
DPPKTHVTHHPVFDYEATLRCWALGFYPAEIILTWQRDGED
QTQDVELVETRPAGDGTFQKWAAVVVPSGEEQRYTCHVQHE
GLPEPLMLRW,
Factor Xa restriction site:
(SEQ ID NO: 10)
IEGRTGTKLGP
SpotTag:
(SEQ ID NO: 11)
PDRVRAVSHWSSC
Myc tag:
(SEQ ID NO: 12)
EQKLISEEDL
His tag:
(SEQ ID NO: 13)
HHHHHH*
Spacer sequence: e.g.
(SEQ ID NO: 14)
NSAVD
or
GS

Most preferred exemplary peptide antigens which can be part of the recombinant polypeptides of the invention are as follows:

| | | SED | |
|---|---|---|---|
| | peptide | ID | |
| Name | antigen | NO | Remarks |
| AIM3_b2mLP_ | ALWGP | (SEQ | preferably used in |
| hINS_15_ | DPAAA | ID | recombinant polypeptides |
| A2G_Spt | | NO: | containing HLA-A2 alpha1 |
| | | 2) | and 2 domains |
| AIM3_b2mLP_ | ILKDF | (SEQ | preferably used in |
| hZnT8_266_ | SILL | ID | recombinant polypeptides |
| A2G_Spt | | NO: | containing HLA-A2 alpha1 |
| | | 22) | and 2 domains |
| AIM3_b2mLP_ | EWESN | (SEQ | preferably used in |
| hGAD65_573_ | GQPE | ID | recombinant polypeptides |
| A2G_Spt | | NO: | containing HLA-A2 alpha1 |
| | | 23) | and 2 domains |
| AIM3_b2mLP_ | KIADP | (SEQ | preferably used in |
| hZnT8_245_ | ICTF | ID | recombinant polypeptides |
| A2G_Spt | | NO: | containing HLA-A2 alpha1 |
| | | 24) | and 2 domains |

Further preferred exemplary peptide antigens which can be part of the recombinant polypeptides of the invention are as follows:

| | | SED | |
|---|---|---|---|
| | peptide | ID | |
| Name | antigen | NO | Remark |
| AIM3_b2mLP_ | LALWG | (SEQ | preferably used in |
| hINS_14_ | PDPAA | ID | recombinant |
| A2G_Spt | | NO: | polypeptides containing |
| | | 25) | HLA-A2 alpha1 and 2 |
| | | | domains |
| AIM3_b2mLP_ | WYIPP | (SEQ | preferably used in |
| hGAD65_507_ | SLRTL | ID | recombinant |
| HLAG_Spt | | NO: | polypeptides containing |
| | | 26) | HLA-G alpha1 and 2 |
| | | | domains |
| AIM3_b2mLP_ | RMMEY | (SEQ | preferably used in |
| hGAD65_536_ | GTTM | ID | recombinant |
| A2G_Spt | | NO: | polypeptides containing |
| | | 27) | HLA-A2 alpha1 and 2 |
| | | | domains |

Example for a Recombinant Polypeptide of the Invention (with the Optional Leader Peptide):

MSRSVALAVLALLSLSGLEAALWGPDPAAAGGCGASGGGGSGGGG
SIQRTPKIQVYSRHPAENGKSNFLNCYVSGFHPSDIEVDLLKNGE
RIEKVEHSDLSFSKDWSFYLLYYTEFTPTEKDEYACRVNHVTLSQ
PKIVKWDRDMGGGGSGGGGSGGGGSGGGGSGSHSMRYFFTSVSRP
GRGEPRFIAVGYVDDTQFVRFDSDAASQRMEPRAPWIEQEGPEYW
DGETRKVKAHSQTHRVDLGTLRGCYNQSEAGSHTVQRMYGCDVGS
DWRFLRGYHQYAYDGKDYIALKEDLRSWTAADMAAQTTKHKWEAA
HVAEQLRAYLEGTCVEWLRRYLENGKETLQRTDPPKTHVTHHPVF
DYEATLRCWALGFYPAEIILTWQRDGEDQTQDVELVETRPAGDGT
FQKWAAVVVPSGEEQRYTCHVQHEGLPEPLMLRWSKEGDGGIMSV
RESRSLSEDLGSPDRVRAVSHWSSC*
(SEQ ID NO: 15; note that
the asterisk denotes the stop codon)

Note that the sequence of the peptide antigen of the above full length recombinant polypeptide can be substituted by any peptide antigen sequence in accordance with the invention, i.e. by any peptide antigen presented by said recombinant polypeptide, wherein the peptide antigen is a peptide of human proinsulin or human insulin, human Glutamate decarboxylase 65, human islet amyloid polypeptide or human Zinc transporter 8. That is, recombinant polypeptides of the invention may consist of a sequence consisting of a peptide antigen which is a peptide of human proinsulin or human insulin, human Glutamate decarboxylase 65, human islet amyloid polypeptide or human Zinc transporter 8 (e.g., any one of the peptide antigens of SEQ ID NOs: 2, 22, 23, 24, 25, 26 and 27), followed by the sequence of

GCGASGGGGSGGGGSIQRTPKIQVYSRHPAENGKSNFLNCYVSGF
HPSDIEVDLLKNGERIEKVEHSDLSFSKDWSFYLLYYTEFTPTEK
DEYACRVNHVTLSQPKIVKWDRDMGGGGSGGGGSGGGGSGGGGSG
SHSMRYFFTSVSRPGRGEPRFIAVGYVDDTQFVRFDSDAASQRME
PRAPWIEQEGPEYWDGETRKVKAHSQTHRVDLGTLRGCYNQSEAG
SHTVQRMYGCDVGSDWRFLRGYHQYAYDGKDYIALKEDLRSWTAA
DMAAQTTKHKWEAAHVAEQLRAYLEGTCVEWLRRYLENGKETLQR
TDPPKTHVTHHPVFDYEATLRCWALGFYPAEIILTWQRDGEDQTQ
DVELVETRPAGDGTFQKWAAVVVPSGEEQRYTCHVQHEGLPEPLM
LRWSKEGDGGIMSVRESRSLSEDLGSPDRVRAVSHWSSC*
(SEQ ID NO: 16; note that the asterisk
denotes the stop codon)

These recombinant polypeptides of the invention may also contain the optional leader peptide as exemplified above.

The receptors ILT2 (also known as LILRB1) and ILT4 (also known as LILRB2) are known in the art. Preferred sequences of these receptors in accordance with the invention are as follows:

ILT2:
(SEQ ID NO: 17)
MTPILTVLICLGLSLGPRTHVQAGHLPKPTLWAEPGSVITQGSPV
TLRCQGGQETQEYRLYREKKTALWITRIPQELVKKGQFPIPSITW
EHAGRYRCYYGSDTAGRSESSDPLELVVTGAYIKPTLSAQPSPVV
NSGGNVILQCDSQVAFDGFSLCKEGEDEHPQCLNSQPHARGSSRA
IFSVGPVSPSRRWWYRCYAYDSNSPYEWSLPSDLLELLVLGVSKK
PSLSVQPGPIVAPEETLTLQCGSDAGYNRFVLYKDGERDFLQLAG
AQPQAGLSQANFTLGPVSRSYGGQYRCYGAHNLSSEWSAPSDPLD
ILIAGQFYDRVSLSVQPGPTVASGENVTLLCQSQGWMQTFLLTKE
GAADDPWRLRSTYQSQKYQAEFPMGPVTSAHAGTYRCYGSQSSKP
YLLTHPSDPLELVVSGPSGGPSSPTTGPTSTSGPEDQPLTPTGSD
PQSGLGRHLGVVIGILVAVILLLLLLLLLFLILRHRRQGKHWTST
QRKADFQHPAGAVGPEPTDRGLQWRSSPAADAQEENLYAAVKHTQ
PEDGVEMDTRSPHDEDPQAVTYAEVKHSRPRREMASPPSPLSGEF
LDTKDRQAEEDRQMDTEAAASEAPQDVTYAQLHSLTLRREATEPP
PSQEGPSPAVPSIYATLAIH
ILT4:
(SEQ ID NO: 18)
MTPIVTVLICLGLSLGPRTHVQTGTIPKPTLWAEPDSVITQGSPV
TLSCQGSLEAQEYRLYREKKSASWITRIRPELVKNGQFHIPSITW
EHTGRYGCQYYSRARWSELSDPLVLVMTGAYPKPTLSAQPSPVVT
SGGRVTLQCESQVAFGGFILCKEGEEEHPQCLNSQPHARGSSRAI
FSVGPVSPNRRWSHRCYGYDLNSPYVWSSPSDLLELLVPGVSKKP
SLSVQPGPVVAPGESLTLQCVSDVGYDRFVLYKEGERDLRQLPGR
QPQAGLSQANFTLGPVSRSYGGQYRCYGAHNLSSECSAPSDPLDI
LITGQIRGTPFISVQPGPTVASGENVTLLCQSWRQFHTFLLTKAG
AADAPLRLRSIHEYPKYQAEFPMSPVTSAHAGTYRCYGSLNSDPY
LLSHPSEPLELVVSGPSMGSSPPPTGPISTPAGPEDQPLTPTGSD
PQSGLGRHLGVVIGILVAVVLLLLLLLLLFLILRHRRQGKHWTST
QRKADFQHPAGAVGPEPTDRGLQWRSSPAADAQEENLYAAVKDTQ
PEDGVEMDTRAAASEAPQDVTYAQLHSLTLRRKATEPPPSQEREP
PAEPSIYATLAIH

The sequences of human proinsulin and human insulin, human Glutamate decarboxylase 65, human islet amyloid polypeptide and human Zinc transporter 8 are known in the art. Preferred amino acid sequences of these proteins are as follows:
human proinsulin and human insulin:
full-length human insulin (consisting of 24 aa signal peptide, 30 aa B-chain, 31 aa C-peptide, 21 aa A chain) reference sequence >sp|P01308|INS_HUMAN Insulin OS=Homo sapiens OX=9606 GN=INS PE=1 SV=1

(SEQ ID NO: 19)
MALWMRLLPLLALLALWGPDPAAAFVNQHLCGSHLVEALYLVCGE
RGFFYTPKTRREAEDLQVGQVELGGGPGAGSLQPLALEGSLQKRG
IVEQCCTSICSLYQLENYCN

proinsulin (after cleavage of signal peptide)

(SEQ ID NO: 28)
FVNQHLCGSHLVEALYLVCGERGFFYTPKTRREAEDLQVGQVELG
GGPGAGSLQPLALEGSLQKRGIVEQCCTSICSLYQLENYCN

mature insulin (B chain and A chain)

(SEQ ID NO: 29)
FVNQHLCGSHLVEALYLVCGERGFFYTPKT
(B chain)
(SEQ ID NO: 30)
LQKRGIVEQCCTSICSLYQLENYCN
(A chain)

human Glutamate decarboxylase 65 (GAD65):
   NP_001127838.1 glutamate decarboxylase 2 [Homo sapiens]

MASPGSGFWSFGSEDGSGDSENPGTARAWCQVAQKFTGGIGNKLC
ALLYGDAEKPAESGGSQPPRAAARKAACACDQKPCSCSKVDVNYA
FLHATDLLPACDGERPTLAFLQDVMNILLQYVVKSFDRSTKVIDF
HYPNELLQEYNWELADQPQNLEEILMHCQTTLKYAIKTGHPRYFN
QLSTGLDMVGLAADWLTSTANTNMFTYEIAPVFVLLEYVTLKKMR
EIIGWPGGSGDGIFSPGGAISNMYAMMIARFKMFPEVKEKGMAAL
PRLIAFTSEHSHFSLKKGAAALGIGTDSVILIKCDERGKMIPSDL
ERRILEAKQKGFVPFLVSATAGTTVYGAFDPLLAVADICKKYKIW
MHVDAAWGGGLLMSRKHKWKLSGVERANSVTWNPHKMMGVPLQCS
ALLVREEGLMQNCNQMHASYLFQQDKHYDLSYDTGDKALQCGRHV
DVFKLWLMWRAKGTTGFEAHVDKCLELAEYLYNIIKNREGYEMVF
DGKPQHTNVCFWYIPPSLRTLEDNEERMSRLSKVAPVIKARMMEY
GTTMVSYQPLGDKVNFFRMVISNPAATHQDIDFLIEEIERLGQDL
(SEQ ID NO: 31; note that glutamate
decarboxylase 2/GAD2 is a synonym for GAD65)

human Islet amyloid polypeptide:
   NP_000406.1 islet amyloid polypeptide preproprotein [Homo sapiens]

(SEQ ID NO: 32)
MGILKLQVFLIVLSVALNHLKATPIESHQVEKRKCNTATCATQRL
ANFLVHSSNNFGAILSSTNVGSNTYGKRNAVEVLKREPLNYLPL

human Zinc transporter 8:
   NP_776250.2 zinc transporter 8 isoform a [Homo sapiens]

(SEQ ID NO: 33)
MEFLERTYLVNDKAAKMYAFTLESVELQQKPVNKDQCPRERPEEL
ESGGMYHCHSGSKPTEKGANEYAYAKWKLCSASAICFIFMIAEVV
GGHIAGSLAVVTDAAHLLIDLTSFLLSLFSLWLSSKPPSKRLTFG
WHRAEILGALLSILCIWVVTGVLVYLACERLLYPDYQIQATVMII
VSSCAVAANIVLTWVLHQRCLGHNHKEVQANASVRAAFVHALGDL
FQSISVLISALIIYFKPEYKIADPICTFIFSILVLASTITILKDF
SILLMEGVPKSLNYSGVKELILAVDGVLSVHSLHIWSLTMNQVIL
SAHVATAASRDSQWVRREIAKALSKSFTMHSLTIQMESPVDQDPD
CLFCEDPCD

The present invention is further illustrated by the following non-limiting examples:

### EXAMPLES

### Example 1

### Methods for Producing Recombinant Polypeptides of the Invention

Expi-293F cells (Thermo Fisher), grown in Expi-293™ expression medium (Thermo Fisher): transfection of 1 μg DNA into 2.5×10⁶ cells/ml using the Expifectamine™ 293 Transfection kit (Thermo Fisher) using Opti-MEM (Thermo Fisher) for complexation of DNA with Expifectamine, after 18-20 h, addition of enhancer according to the protocol, harvesting of the supernatant after 4-6 days (37° C., 8% CO₂, humidified incubator), 19 mm² orbital shaker 125 rpm
Spot-tag protein purification: equilibration of Spot-Cap resin: transfer of desired slurry amount into an appropriate tube, sediment beads by centrifugation (4° C., 4 min, 2500 g), remove & discard supernatant, add 10 bed volumes PBS (cold) to beads, invert to mix, sediment beads by centrifugation (4° C., 4 min, 2500 g), remove & discard supernatant, repeat 2 times
Add required volume beads to supernatant, incubate ON, 4° C. on a rotator, wash beads by repeated centrifugation (4° C., 4 min, 2500 g), and removal of supernatant
Prepare a 500 μM Spot-peptide solution in PBS, remove the supernatant, incubate with ⅓rd of the spot-peptide solution for 5-10 min

Sediment beads by centrifugation. Use Amicon Ultra-4 centrifugal filters (15 kDa cutoff) for Protein concentration and spot-peptide removal with 15 kDa Amicon cutoff columns

Rinse the Amicon Ultra-4 centrifugal filters (15 kDa cutoff) with PBS followed by 0.1 N NaOH (centrifugation at 4000 g, 4° C.) to remove trace amounts of glycerine.

### ELISPOT:

### 1) Cell Culture

### A) PBMC Isolation (Under a Laminar Flow Hood)

To isolate peripheral blood mononuclear cells (PBMC), a density centrifugation was performed with white blood cells from a leukocyte reduction chamber and density gradient medium (e.g. Ficoll, or ROTI Sep 1077).

Cells were centrifuged for 20 min at 1200×g without brake followed by collection of the interphase ring that was washed with 1×PBS (5 min, 300×g). PBMC were frozen till further use.

### B) PBMC Pulsing (Under a Laminar Flow Hood)

PBMCs were thawed 1 day prior to PBMC pulsing (d−1) and kept over night in 5 ml X-VIVO 15 medium containing 5% human AB serum in a well of a 6 well plate at 37° C.

On the next day (d0) cells were counted and resuspended in X-VIVO 15 complete medium (5% hAB serum & cytokine cocktail: 20 ng/ml hIL-2, 20 ng/ml hGM-CSF, 10 ng/ml hIL-4 & 10 ng/ml hTGF-b1) at a cell density of 3×10⁶ cells/ml.

For experiments, 3×10⁶ cells were seeded in the respective wells of a 12-well plate with a final volume of 1000 μl X-VIVO complete medium with cytokine cocktail and

5 μg/ml of an AIM Bio molecule or the respective controls.

On day 3, 1 ml complete medium (with cytokines) was added, on day 6, a second pulse with 5 μg/ml of a recombinant polypeptide of the invention or a surrogate thereof (collectively referred to as “AIM Bio” molecule) was performed (after removing medium). On days 7, 10 & 12, 1 ml complete medium (with cytokines) was added.

### Required:

X-VIVO 15 medium+5% human AB serum
X-VIVO 15 complete medium: X-VIVO 15 medium+2% human AB serum supplemented with cytokine cocktail: 10 ng/ml TGF-b1, 10 ng/ml IL-4, 20 ng/ml IL-2, 20 ng/ml GM-CSF
6 well plate
12 well plate

### 2) ELISPOT

### Laminar Flow Hood

On day 13, ELISPOT plates were coated using anti-hIL10 (clone 9D-7, 1:500 dilution in PBS, sterile filtered) and aIL10 (10G8-biotin) and on day 14, 200,000 cells were seeded per well on the ELISPOT plates in duplicates, including negative controls (cells plus PBS) and a positive control (e.g. LPS).

The PFDF membrane was activated with 50 μl/well EtOH (35% v/v) for 1 min followed by 5× washing with 200 μl distilled sterile water. Plate was coated with 100 μl/well antibody solution at 4° C. over night. On the next day, unbound coating antibody was removed, 5 washing steps were performed with 200 μl PBS and 200 μl blocking buffer (X-VIVO 15 5% hAB serum) was added and the plate incubated for 30 min-2 h at room temperature.

The respective antigenic peptide (e.g. MOG 157) in DMSO or DMSO as a control were prepared, and a final amount of 5 μg peptide/ml was added to the final volume of 100 μl/well. 150,000 cells were seeded per well in X-VIVO 15 medium with 5% human AB serum. Blocking buffer (X VIVO 15 medum+5% hAB serum) was carefully removed, and medium with PBS as negative control and stimulants (5 μg/ml total volume in each well) were added to the other wells and incubated at 37° C. over night.

### Outside the Laminar Flow Hood

Secondary antibody was prepared: 1 μg/ml aIL-10-biotinylated antibody in 0.5% BSA/1×PBS (1:1000 dilution) and horseradish peroxidase-conjugated streptavidin (1:750 in 0.5% BSA/PBS), tetramethylbenzidine solution was filtered using a 0.45 μm filter and stored at 4° C. till use.

Cell supernatant was removed and 5× washed using 100 μl PBS. Last excess buffer was removed using paper towels.

25 μl diluted HRP-streptavidin (1:750) was added per well and incubated for 1 h at room temperature in the dark followed by 5 washing steps using sterile 1×PBS.

100 μl of filtered TMB substrate was added per well for 15-25 min till blue sports developed. Reaction was stopped by washing the wells thoroughly with tapped water.

Plastic underdrains of the plates was removed and the bottom and sides of the plates were washed with tap water and dried.

Plates were read out using an ImmunoSpot S6 Ultra-V Analyzer (Cellular Technology Limited), analysed in Excel and graphs/statistics were done in Graphad Prism.

### Required:

Capture antibodies: anti-hIL10 (Clone: 9D-7, Mabtech #3430-3-250; 1:500 dilution), anti-hIL10-biotinylated (Mabtech, #3430-6-250)
1×PBS (sterile)
35% EtOH (v/v)
Blocking buffer: X-vivo 5% hAB serum (sterile) [blocking is done in the same medium as cell culture]
Dilution buffer: 0.5% BAS in PBS
Washing buffer: 1×PBS
Medium: for T cells, X-VIVO 15 medium (Lonza)
Filter syringe: Millex GV
ELISPOT PVDF plate (#MSIP4510, Millipore)
TMB substrate

### Example 2: Surrogates of Recombinant Polypeptides of the Invention Induce IL10 Secreting Treg in Mice

Wild type black 6 mice were injected with 100 μg recombinant polypeptides (also referred to as “AIMBio”) having the following sequences,

Ova_KbG
(SEQ ID NO: 34)
SIINFEKLGCGASGGGGSGGGGSIQRTPKIQVYSRHPAENGKSNF
LNCYVSGFHPSDIEVDLLKNGERIEKVEHSDLSFSKDWSFYLLYY
TEFTPTEKDEYACRVNHVTLSQPKIVKWDRDMGGGGSGGGGSGGG
GSGGGGSGPHSLRYFVTAVSRPGLGEPRYMEVGYVDDTEFVRFDS
DAENPRYEPRARWMEQEGPEYWERETQKAKGNEQSFRVDLRTLLG
CYNQSKGGSHTIQVISGCEVGSDGRLLRGYQQYAYDGCDYIALNE
DLKTWTAADMAALITKHKWEQAGEAERLRAYLEGTCVEWLRRYLK
NGNATLLRTDPPKTHVTHHPVFDYEATLRCWALGFYPAEIILTWQ
RDGEDQTQDVELVETRPAGDGTFQKWAAVWPSGEEQRYTCHVQHE
GLPEPLMLRWSKEGDGGIMSVRESRSLSEDLGSPDRVRAVSHWSS
C
and
Gp34_KbG
(SEQ ID NO: 35)
AVYNFATMGCGASGGGGSGGGGSIQRTPKIQVYSRHPAENGKSNF
LNCYVSGFHPSDIEVDLLKNGERIEKVEHSDLSFSKDWSFYLLYY
TEFTPTEKDEYACRVNHVTLSQPKIVKWDRDMGGGGSGGGGSGGG
GSGGGGSGPHSLRYFVTAVSRPGLGEPRYMEVGYVDDTEFVRFDS
DAENPRYEPRARWMEQEGPEYWERETQKAKGNEQSFRVDLRTLLG
CYNQSKGGSHTIQVISGCEVGSDGRLLRGYQQYAYDGCDYIALNE
DLKTWTAADMAALITKHKWEQAGEAERLRAYLEGTCVEWLRRYLK
NGNATLLRTDPPKTHVTHHPVFDYEATLRCWALGFYPAEIILTWQ
RDGEDQTQDVELVETRPAGDGTFQKWAAVVVPSGEEQRYTCHVQH
EGLPEPLMLRWSKEGDGGIMSVRESRSLSEDLGSPDRVRAVSHWS
SC,

for inducing tolerance towards an OVA peptide or an viral Gp34 peptide, respectively. The Gp34 peptide is a well-characterized T cell epitope derived from Lymphocytic Choriomeningitis virus (LCMV) Glycoprotein. While this antigen was traditionally named Gp33, the epitope presented on H2-K^{b} was later found to comprise just amino acids 34-41. (An epitope beginning at amino acid 33 is, in contrast, presented on H2-K^{d}.) Therefore, we call the H2-K^{b} epitope Gp34, which is in line with the most recent recommendations. Still, there is an ambiguous use of the Gp33 and Gp34 nomenclature in the literature. The first 8 amino acids of SEQ ID NO: 35 show the correct sequence (AVYNFA™; SEQ ID NO: 58). After 2 weeks, mice were sacrificed, and splenocytes re-challenged either with the matching or a mismatching peptide. IL-10 secreting cells were quantified by ELIspot. The results are shown in FIG. 3.

### Example 3: Surrogates of Recombinant Polypeptides of the Invention Selectively Prevent CD8+ T-Cell Driven EAE in Mice

As described in (Na et al, Brain. 2008 September; 131(Pt 9):2353-65.), the adoptive transfer of CD8⁺ OT-I T cells that recognize an ovalbumin epitope in the context of H2-K^{b} into mice which express ovalbumin in oligodendrocytes leads to experimental autoimmune encephalomyelitis which recapitulates many MS and late stage NMO symptoms. In this animal model, a single injection of 500 μg of recombinant polypeptides surrogate molecules (also referred to as “AIMBio”) that induce tolerance towards the targeted ovalbumin epitope almost completely prevented EAE symptoms, while a surrogate molecule presenting a control peptide hat no significant protective effects (FIG. 4). The sequences of the recombinant polypeptide surrogate molecules were as follows:

Mog44_DbG
(SEQ ID NO: 36)
FSRVVHLYRNGGCGASGGGGGGGGSIQRTPKIQVYSRHPAENGKS
NFLNCYVSGFHPSDIEVDLLKNGERIEKVEHSDLSFSKDWSFYLL
YYTEFTPTEKDEYACRVNHVTLSQPKIVKWDRDMGGGGSGGGGSG
GGGSGGGGSGPHSMRYFETAVSRPGLEEPRYISVGYVDNKEFVRF
DSDAENPRYEPRAPWMEQEGPEYWERETQKAKGQEQWFRVSLRNL
LGCYNQSAGGSHTLQQMSGCDLGSDWRLLRGYLQFAYEGRDYIAL
NEDLKTWTAADMAAQITRRKWEQSGAAEHYKAYLEGECVEWLHRY
LKNGNATLLRTDPPKTHVTHHPVFDYEATLRCWALGFYPAEIILT
WQRDGEDQTQDVELVETRPAGDGTFQKWAAVVVPSGEEQRYTCHV
QHEGLPEPLMLRWSKEGDGGIMSVRESRSLSEDLGSPDRVRAVSH
WSSC
Mog37_DbG
(SEQ ID NO: 37)
VGWYRSPFSRGCGASGGGGSGGGGSIQRTPKIQVYSRHPAENGKS
NFLNCYVSGFHPSDIEVDLLKNGERIEKVEHSDLSFSKDWSFYLL
YYTEFTPTEKDEYACRVNHVTLSQPKIVKWDRDMGGGGSGGGGSG
GGGSGGGGSGPHSMRYFETAVSRPGLEEPRYISVGYVDNKEFVRF
DSDAENPRYEPRAPWMEQEGPEYWERETQKAKGQEQWFRVSLRNL
LGCYNQSAGGSHTLQQMSGCDLGSDWRLLRGYLQFAYEGRDYIAL
NEDLKTWTAADMAAQITRRKWEQSGAAEHYKAYLEGECVEWLHRY
LKNGNATLLRTDPPKTHVTHHPVFDYEATLRCWALGFYPAEIILT
WQRDGEDQTQDVELVETRPAGDGTFQKWAAVVVPSGEEQRYTCHV
QHEGLPEPLMLRWSKEGDGGIMSVRESRSLSEDLGSPDRVRAVSH
WSSC

### Example 4: Some Surrogates of Recombinant Polypeptides of the Invention Selectively Prevent CD4⁺ T Cell Driven EAE in Mice

At the day of the 33 μg or 100 μg recombinant polypeptide of the invention surrogate molecule (“AIM Bio”) i.p. injection, 100 μl MOG35-55 peptide/CFA (Complete Freund's Adjuvance; final concentration Mycobacterium tuberculosis H37RA and peptide each 1 mg/ml) emulsion were injected each left and right s.c. into the flank and 250 ng pertussis toxin (in 200 μl PBS) intraperitoneally. A second pertussis toxin injection was given 3 days later. In this animal model, a single injection AIM Bio surrogate molecules that induce tolerance towards the a Mog epitope (Mog44_Kb_G) significantly reduced EAE symptoms, while a surrogate molecule presenting a control peptide (Gp34) or a non-functional Mog peptide (Mog37) hat no significant protective effects (FIG. 5). In this model Mog44 AIM Bio also prevented inflammation and CD8 T cell infiltration in the spinal cord (FIG. 6). The sequences of the recombinant polypeptide surrogate molecules were shown in Example 3.

In this model Mog44 AIM Bio also completely prevented the formation of MOG-specific autoantibodies in the serum as tested by ELISA (FIG. 7). This is a strong indicator that the recombinant polypeptides of the invention are effective therapeutics in NMO, which are often characterized by antibody responses against human aquaporin 4, and in other autoimmune diseases such as Type 1 diabetes, the pathology of which is also characterized by the presence of autoantibodies. Thus, the patient population is defined by a common autoimmune-related antigen. Certain MHC molecules are also associated with NMO.

Mog-reactive antibodies in sera of AIM Bio (33 or 100 μg) treated mice were detected via standard ELISA protocol, with 3 washes in between each step. Briefly, ELISA plates were coated with 10 μg/ml Mog35-55 peptide, blocked with PBS 1% BSA, before mouse sera diluted 1:25 in PBS 1% BSA were added for 1 h. Anti-mouse IgG-HRP or anti-mouse heavy and light chain—HRP antibodies diluted 1:5000 were used for detection.

### Example 5: Human Recombinant Polypeptide Candidates of the Invention for T1D

The recombinant polypeptides of the invention are newly developed protein complexes derived from the pregnancy-associated immunosuppressive MHC molecule HLA-G. It is likely that HLA-G enables an embryo to influence the maternal immune system to tolerate embryonic antigens but further antagonize antigens from pathogens. The recombinant polypeptides of the invention containing variable peptides were able to selectively eliminate peptide-specific cytotoxic effector T cells as well as induce peptide-specific regulatory T cells in the test tube.

T1D autoantigens in accordance with the invention include (pro-)insulin (INS), Glutamate decarboxylase 65 (GAD65), islet amyloid polypeptide (IAPP) or Zinc transporter 8 (ZNT8).

FIG. 8 shows a list of the human T1D recombinant polypeptide candidates.

The inventors' findings show that single-chain proteins containing an INS, GAD65, IAPP or ZNT8 peptide antigen and a HLA-G alpha 3 domain can induce tolerogenic T cells in healthy donors. Thus, CD8 Treg were upregulated by at least 30% in 75% of all healthy blood donors (FIG. 9).

In correlation with the in vivo experiment shown here, it is plausible that these constructs suppress CD8 T cell mediated and antibody mediated responses directed against islet cell antigens in patients.

### Example 6: Further Proof-of-Principle of Stability and Effects of the Recombinant Polypeptides of the Invention

Additionally, the inventors set out to obtain and test recombinant polypeptides having the general structure of the recombinant polypeptides of the invention but containing various different peptide antigens, in order to obtain further proof-of-principle that recombinant polypeptides of the invention and surrogates thereof are stable and efficacious. As shown in FIGS. 10 and 11, respectively, the tested recombinant polypeptides are stable during freeze-thawing and storage and are thermally stable. Further, they induce Treg in a dose-dependent manner (FIG. 12) and inhibit T cell lysis in a dose-dependent manner (FIG. 13). Effects of the recombinant polypeptides on the serum cytokine profile in EAE-ODC Ova mice are shown in FIG. 14. There is an induction of IL-10 and possibly IL-4, both known to be immunosuppressive cytokines downregulating immune responses in inflammatory settings. This requires an HLA-G alpha3 domain plus a cognate peptide. IL-2 seems to be induced in response to presenting the cells with a cognate peptide that is irrespective of the alpha3 domain. IL-2 is needed for T cell activation and survival.

Further, the high melting temperatures shown in FIG. 16 confirm good protein stability of the recombinant polypeptides of the invention for therapeutic use. Additionally, the data in FIG. 17 indicate that T1D single chain MHC Ib molecules (recombinant polypeptides of the invention) can be purified and stored and are resistant to freeze-thaw cycles.

### Example 7: As Indicated in FIG. 15, there is an Upregulation of CD8 Treg in in PBMCs of Healthy Blood Donors by a Recombinant Polypeptide of the Invention

In vitro Treg induction mediated by peptide-HLA-G containing constructs (AIM Biologicals) was carried out as follows: PBMCs from healthy donors were purified via density centrifugation performed on white blood cells from a leukocyte reduction chamber using Ficoll. Cells were centrifuged for 20 min at 1200×g without brake followed by collection of the interphase ring that was washed with 1×PBS (5 min, 300×g). PBMC were frozen till further use.

PBMCs were thawed 1 day prior to PBMC pulsing (d−1) and kept over night in 5 ml X-VIVO 15 medium containing 5% human AB serum in a well of a 6 well plate at 37° C.

On the next day (d0), cells were counted and resuspended in X-VIVO 15 complete medium (5% hAB serum & cytokine cocktail: 20 ng/ml hIL-2, 20 ng/ml hGM-CSF, 10 ng/ml hIL-4 & 10 ng/ml hTGF-b1) at a cell density of 3×10⁶ cells/ml. For experiments, 3×10⁶ cells were seeded in the respective wells of a 12-well plate with a final volume of 1000 μl X-VIVO complete medium with cytokine cocktail and 5 μg/ml of an AIM Bio molecule or the respective controls.

On day 3, 1 ml complete medium (with cytokines) was added, on day 6, a second pulse with 5 μg/ml AIM Bio molecule was performed (after removing medium). On days 7, 10 & 12, 1 ml complete medium (with cytokines) was added.

On day 13, ELISpot plate PVDF membrane was activated with 50 μl/well EtOH (35% v/v) for 1 min followed by 5× washing with 200 μl distilled sterile water. Plate was coated with 100 μl/well anti-hIL10 (clone 9D-7, 1:500 dilution in PBS, sterile filtered) at 4° C. over night. On the next day, unbound coating antibody was removed, 5 washing steps were performed with 200 μl PBS and 200 μl blocking buffer (X-VIVO 15 5% hAB serum) was added and the plate incubated for 30 min-2 h at room temperature. Day 14, 200,000 cells were seeded per well on the ELISpot plates in duplicates, including negative controls (cells plus PBS) and a positive control (e.g. LPS) for 48 h. Secondary antibody was prepared: 1 μg/ml aIL-10-biotinylated antibody in 0.5% BSA/1×PBS (1:1000 dilution) and horseradish peroxidase-conjugated streptavidin (1:750 in 0.5% BSA/PBS), tetramethylbenzidine solution was filtered using a 0.45 μm filter and stored at 4° C. till use. Cell supernatant was removed and 5× washed using 100 μl PBS. Last excess buffer was removed using paper towels. 25 μl diluted HRP-streptavidin (1:750) was added per well and incubated for 1 h at room temperature in the dark followed by 5 washing steps using sterile 1×PBS. 100 μl of filtered TMB substrate was added per well for 15-25 min till blue sports developed. Reaction was stopped by washing the wells thoroughly with water. Plastic underdrains of the plates were removed and the bottom and sides of the plates were also washed with tap water and dried.

Some recombinant polypeptides of the invention induced at least 30% more IL-10 secreting T reg in PBMCs of ˜75% of all healthy blood donors.

### INDUSTRIAL APPLICABILITY

The pharmaceutical compositions, polypeptides, nucleic acids, cells, and products for use in the invention are industrially applicable. For example, they can be used in the manufacture of, or as, pharmaceutical products.

1. A recombinant polypeptide capable of presenting a peptide antigen, the recombinant polypeptide comprising, in an N- to C-terminal order,
i) a peptide antigen presented by said recombinant polypeptide, wherein the peptide antigen is a peptide of human proinsulin or human insulin, human Glutamate decarboxylase 65, human islet amyloid polypeptide or human Zinc transporter 8;
ii) optionally a linker sequence;
iii) optionally a sequence of a human polypeptide domain comprising a sequence of a human 132 microglobulin, or an amino acid sequence at least 90% identical to the amino acid sequence of human 132 microglobulin represented by SEQ ID NO: 5;
iv) optionally a linker sequence;
v) optionally an [alpha]1 domain of an MHC molecule;
vi) optionally an [alpha]2 domain of an MHC molecule;
vii) an [alpha]3 domain of an MHC class Ib molecule or a derivative of an [alpha]3 domain of an MHC class Ib molecule, said derivative being capable of binding to ILT2 or ILT4;
viii) optionally a protease cleavage site;
ix) optionally a spacer sequence; and
x) optionally an affinity tag.

2. The recombinant polypeptide according to claim 1, wherein said peptide antigen according to i) is 7 to 11 amino acids in length, preferably 8-10 amino acids in length.

3. The recombinant polypeptide according to claim 1 or 2, wherein said peptide antigen according to i) consists of an amino acid sequence selected from the group consisting of the amino acid sequences of SEQ ID NO: 2, SEQ ID NO: 22, SEQ ID NO: 23, SEQ ID NO: 24, SEQ ID NO: 25, SEQ ID NO: 26 and SEQ ID NO: 27.

4. The recombinant polypeptide according to any one of the preceding claims, wherein said peptide antigen consists of an amino acid sequence selected from the group consisting of the amino acid sequences of SEQ ID NO: 2, SEQ ID NO: 22, and SEQ ID NO: 23.

5. The recombinant polypeptide according to any one of claims 1-3, wherein said peptide antigen is a peptide antigen of human proinsulin or human insulin and preferably consists of an amino acid sequence selected from the group consisting of SEQ ID NO: 2 and SEQ ID NO: 25.

6. The recombinant polypeptide according to any one of claims 1-3, wherein said peptide antigen is a peptide antigen of human Glutamate decarboxylase 65 and preferably consists of an amino acid sequence selected from the group consisting of SEQ ID NO: 23, SEQ ID NO: 26 and SEQ ID NO: 27.

7. The recombinant polypeptide according to any one of claims 1-3, wherein said peptide antigen is a peptide antigen of human Glutamate decarboxylase 65 and consists of the amino acid sequence of SEQ ID NO: 26.

8. The recombinant polypeptide according to any one of claims 1-3, wherein said peptide antigen is a peptide antigen of human Zinc transporter 8 and preferably consists of an amino acid sequence selected from the group consisting of SEQ ID NO: 22 and SEQ ID NO: 24.

9. The recombinant polypeptide according to any one of claims 1-3, wherein said peptide antigen is a peptide antigen of human islet amyloid polypeptide.

10. The recombinant polypeptide according to any one of the preceding claims, wherein said [alpha]1 domain according to (v) and said [alpha]2 domain according to (vi) are from a human MHC class Ia molecule or from a human MHC class Ib molecule.

11. The recombinant polypeptide according to claim 10, wherein said [alpha]1 domain according to (v) and said [alpha]2 domain according to (vi) are from a human MHC class Ia molecule.

12. The recombinant polypeptide according to claim 11, wherein said [alpha]1 domain according to (v) and said [alpha]2 domain according to (vi) are from a human HLA-A2 molecule.

13. The recombinant polypeptide according to claim 10, wherein said [alpha]1 domain according to (v) and said [alpha]2 domain according to (vi) are from a human MHC class Ib molecule.

14. The recombinant polypeptide according to claim 13, wherein said [alpha]1 domain according to (v) and said [alpha]2 domain according to (vi) are from a human HLA-G molecule.

15. The recombinant polypeptide according to any one of the preceding claims, wherein the [alpha] 3 domain of the MHC class Ib molecule according to (vii) is an [alpha]3 domain of human HLA-E, human HLA-F or human HLA-G.

16. The recombinant polypeptide according to any one of the preceding claims, wherein the [alpha] 3 domain of the MHC class Ib molecule according to (vii) is an [alpha]3 domain of human HLA-G.

17. The recombinant polypeptide according to any one of the preceding claims, wherein the [alpha]3 domain or derivative according to (vii) is identical to or has at least 80% amino acid sequence identity, preferably at least 90% amino acid sequence identity, with the [alpha]3 domain having the amino acid sequence of SEQ ID NO: 9 or SEQ ID NO: 21.

18. The recombinant polypeptide according to claim 17, wherein the [alpha]3 domain or derivative according to (vii) is identical to or has at least 92% amino acid sequence identity with the [alpha]3 domain having the amino acid sequence of SEQ ID NO: 9 or SEQ ID NO: 21.

19. The recombinant polypeptide according to claim 17, wherein the [alpha]3 domain or derivative according to (vii) is identical to or has at least 94% amino acid sequence identity with the [alpha]3 domain having the amino acid sequence of SEQ ID NO: 9 or SEQ ID NO: 21.

20. The recombinant polypeptide according to claim 17, wherein the [alpha]3 domain or derivative according to (vii) is identical to or has at least 96% amino acid sequence identity with the [alpha]3 domain having the amino acid sequence of SEQ ID NO: 9 or SEQ ID NO: 21.

21. The recombinant polypeptide according to claim 17, wherein the [alpha]3 domain or derivative according to (vii) is identical to or has at least 98% amino acid sequence identity with the [alpha]3 domain having the amino acid sequence of SEQ ID NO: 9 or SEQ ID NO: 21.

22. The recombinant polypeptide according to claim 17, wherein the [alpha]3 domain or derivative according to (vii) is identical to or has at least 99% amino acid sequence identity with the [alpha]3 domain having the amino acid sequence of SEQ ID NO: 9 or SEQ ID NO: 21.

23. The recombinant polypeptide according to claim 17, wherein the [alpha]3 domain according to (vii) is identical to the [alpha]3 domain having the amino acid sequence of SEQ ID NO: 9 or SEQ ID NO: 21.

24. The recombinant polypeptide according to any one of the preceding claims, wherein the linker sequence according to (ii) and/or the linker sequence according to (iv) comprises the amino acid sequence (GGGGS)n, wherein n is an integer equal to or higher than 1.

25. The recombinant polypeptide according to claim 24, wherein the linker sequence according to (ii) comprises the amino acid sequence (GGGGS)n, and wherein n is an integer selected from the group consisting of 1, 2, 3, 4, 5, 6, 7, 8, 9 and 10 and is preferably selected from the group consisting of 2, 3, 4 and 5.

26. The recombinant polypeptide according to claim 24 or 25, wherein the linker sequence according to (iv) comprises the amino acid sequence (GGGGS)n, and wherein n is an integer selected from the group consisting of 1, 2, 3, 4, 5, 6, 7, 8, 9 and 10 and is preferably selected from the group consisting of 2, 3, 4 and 5.

27. The recombinant polypeptide according to any one of the preceding claims, wherein said sequence of a human polypeptide domain according to (iii) is at least 95% identical to the amino acid sequence of SEQ ID NO: 5, preferably at least 98% identical to the amino acid sequence of SEQ ID NO: 5 and more preferably identical to the amino acid sequence of SEQ ID NO: 5.

28. The recombinant polypeptide according to any one of the preceding claims, wherein said polypeptide is dimeric or multimeric.

29. The recombinant polypeptide according to any one of the preceding claims, wherein the polypeptide comprises or consists of all of the components i) to vii)

30. The recombinant polypeptide according to any one of the preceding claims, wherein the polypeptide does not comprise components viii) to x).

31. The recombinant polypeptide according to any one of claims 1 to 29, wherein the polypeptide comprises or consists of all of the components i) to x).

32. The recombinant polypeptide according to any one of the preceding claims, further comprising an N-terminal secretion signal peptide sequence.

33. The recombinant polypeptide according to any one of claims 1-31, wherein the recombinant polypeptide consists of an amino acid sequence consisting of the following ((a) and (b)) in an N- to C-terminal order:
(a) a peptide antigen selected from the group consisting of the amino acid sequences of SEQ ID NO: 2, SEQ ID NO: 22, SEQ ID NO: 23, SEQ ID NO: 24, SEQ ID NO: 25, SEQ ID NO: 26 and SEQ ID NO: 2, and
(b) the amino acid sequence of SEQ ID NO: 16.

34. The recombinant polypeptide according to any one of the preceding claims, wherein the recombinant polypeptide is soluble.

35. A nucleic acid encoding one or more polypeptides according to any one of the preceding claims.

36. The nucleic acid according to claim 35, wherein the nucleic acid is a vector.

37. A pharmaceutical composition comprising at least one nucleic acid according to claim 35 or 36.

38. A pharmaceutical composition or kit comprising at least one recombinant polypeptide according to any one of claims 1-34.

39. The pharmaceutical composition or kit according to claim 38, wherein the pharmaceutical composition or kit comprises at least two different recombinant polypeptides according to any one of claims 1-34, and wherein each of the different polypeptides comprises a different peptide antigen as defined in any one of claims 3 to 9.

40. The pharmaceutical composition or kit according to claim 38 or 39, wherein the pharmaceutical composition or kit comprises at least the following ((A) to (C)): (A) a recombinant polypeptide wherein said peptide antigen is a peptide antigen of human proinsulin or human insulin; (B) a recombinant polypeptide wherein said peptide antigen is a peptide antigen of human Glutamate decarboxylase 65; (C) a recombinant polypeptide wherein said peptide antigen is a peptide antigen of human Zinc transporter 8; and optionally further comprises (D) a recombinant polypeptide wherein said peptide antigen is a peptide antigen of human islet amyloid polypeptide.

41. The pharmaceutical composition or kit according to any one of claims 38 to 40, wherein the pharmaceutical composition or kit comprises at least three different recombinant polypeptides according to any one of claims 1-34, wherein said peptide antigen of a first recombinant polypeptide of the at least three different recombinant polypeptides consists of an amino acid sequence of SEQ ID NO: 2, wherein said peptide antigen of a second recombinant polypeptide of the at least three different recombinant polypeptides consists of an amino acid sequence of SEQ ID NO: 22, and wherein said peptide antigen of a third recombinant polypeptide of the at least three different recombinant polypeptides consists of an amino acid sequence of SEQ ID NO: 23.

42. A pharmaceutical composition or kit according to any one of claims 37-41, for use in the treatment of type 1 diabetes in a human patient.

43. The pharmaceutical composition or kit for use according to claim 42, wherein the treatment is treatment by immunotherapy.

44. The pharmaceutical composition or kit for use according to any one of claims 42-43, wherein the treatment is by inducing immunological tolerance against human proinsulin and/or human insulin, human Glutamate decarboxylase 65, human islet amyloid polypeptide and/or human Zinc transporter 8.

45. The pharmaceutical composition or kit for use according to any one of claims 42-44, wherein the treatment is for reducing plasma levels of autoantibodies against insulin (insulin autoantibodies IAA), or glutamic acid decarboxylase (GAD-65), or islet antigen-2A (IA-2A), or zinc transporter ZnT8, as assessed by radio-binding assays or non-radioactive electrochemiluminescent antigen-binding assays.

46. The pharmaceutical composition or kit for use according to any one of claims 42-45, wherein the human patient is a patient who had plasma autoantibodies against insulin (insulin autoantibodies IAA), or glutamic acid decarboxylase (GAD-65), or islet antigen-2A (IA-2A), or zinc transporter ZnT8 prior to the start of the treatment.

47. A recombinant host cell comprising a nucleic acid or a vector according to claim 35 or 36 and expressing the recombinant polypeptide according to any one of claims 1-34.

48. A method for obtaining a pharmaceutical composition comprising a polypeptide according to any one of claims 1-34, the method comprising the steps of (a) culturing the recombinant host cell of claim 47 under conditions allowing expression of the recombinant polypeptide from the nucleic acid molecule, (b) recovering the recombinant polypeptide, (c) purifying the recombinant polypeptide, and (d) formulating the recombinant polypeptide into a pharmaceutical composition.
